(19) United States
(12) Patent Application Publication
(10) Pub. No.: US 2026/0131044 A1
(43) Pub. Date: May 14, 2026

(54) FIBERS ELECTROSPINNING INCLUDING MICROFLUIDICS METHOD

(71) Applicants: UNIVERSITY OF TARTU, Tartu (EE); INSTITUTE OF FUNDAMENTAL TECHNOLOGICAL RESEARCH, Warsaw (PL)

(72) Inventors: Georg-Marten LANNO, Tartu (EE); Tomasz KOWALCZYK, Warsaw (PL); Slawomir BLONSKI, Warsaw (PL); Marta PUTRINŠ, Tartu (EE); Tanel TENSON, Tartu (EE); Piotr KORCZYK, Warsaw (PL); Karin KOGERMANN, Tartu (EE)

(21) Appl. No.: 18/695,658

(22) PCT Filed: Sep. 27, 2022
(86) PCT No.: PCT/EP2022/076874

§ 371 Date: Mar. 26, 2024

(30) Foreign Application Priority Data
Sep. 27, 2021 (GB) 2113798.9

(51) Int. Cl. A61L 15/36; A61L 15/22; A61L 15/42; D01D 5/00; D01F 1/10

(52) U.S. Cl. CPC A61L 15/36; A61L 15/225; A61L 15/425; D01D 5/0038; D01D 5/0061; D01F 1/10; A61L 2300/406; A61L 2300/622; A61L 2300/64

(57) ABSTRACT

The present invention relates to methods for the encapsulation of bioactive entities, and formation into solid materials. The method comprises encapsulation of a bioactive entity via microfluidics and electrospinning into polymer fibres. The invention also relates to electrospun polymer fibres comprising bioactive entity-containing hydrophilic and/or gel-forming polymer microparticles embedded within fibres of at least one hydrophobic polymer. The invention also relates to a wound dressing comprising the electrospun fibres.

### FIELD OF THE INVENTION

The present invention relates to methods for the encapsulation of bioactive entities, and formation into solid materials.

### BACKGROUND OF THE INVENTION

Encapsulation is used in a broad range of fields, for example, in bioreactors and medicine. Encapsulation of bioactive entities provides a number of advantages including ease of separation, protection from external conditions and reduced susceptibility to contamination by foreign materials or organisms.

A number of encapsulation techniques are known. The most common encapsulation techniques include spray-drying, emulsifying-crosslinking and coacervation. A problem that arises with these techniques is that the high temperatures or poorly compatible organic agents used in at least one of the production steps can lead to the destruction of sensitive encapsulated bioactive entities as well as toxicity problems associated with residual organic solvents. It would be an advantage to provide new encapsulation techniques which can be used to address these limitations and provide effective encapsulation, especially for sensitive bioactive entities.

Microfluidics can be used to encapsulate and protect bioactive materials including bacteria. Most microfluidic microencapsulation methods for living bacterial cells use hydrophilic polymers, for example PEG-based polymers, Na-alginate or agarose, with mineral oils (Lee et al., Biotechnol. Bioeng., (2010), 107(4), 747-751; Eun et al., ACS Chem. Biol., (2011), 6(3), 260-266). Such systems use bacteria-friendly solvents and therefore the resulting bacteria-containing microparticles may be used to prolong the viability of the bacteria. However, there are also a number of problems associated with microfluidic encapsulation. Challenges include maintaining reasonable levels of cell viability, cell leaching, nutrient and oxygen diffusion and ensuring uniform microbead shape and size distribution. The encapsulation of cells by microfluidics and the use of organic solvents is mentioned in U.S. Pat. No. 9,176,504. However, the conditions used in U.S. Pat. No. 9,176,504 decrease bacterial viability due to the use of a laser pulse for bubble creation.

Electrospinning is a versatile technique used to produce fibres at micro- to nanoscale and can be used to encapsulate living bacterial cells. Hydrophilic polymers are commonly used for such fibre formation (Heunis et al., Probiotics Antimicrob., (2010), 2(1), 46-51; Nagy et al., EXPRESS Polym. Lett., (2014), 8(5), 352-361; Han et al., ACS Appl. Mater. Interfaces, (2018), 10(45), 38799-38806; Sen Kaskin et al., Colloids Surf. B, (2018), 161, 169-176; Škrlec et al., Eur. J. Pharm. Biopharm., (2019), 136, 108-119; Zupančič et al., Pharmaceutics, (2019), 11(9), 483). An advantage of electrospinning includes the production of very thin fibres with large surface areas. However, for applications such as wound healing, electrospinning with hydrophilic water-soluble polymers is not a suitable technique as bacteria or other agents will be released quickly into the wound after application, as the hydrophilic matrix is dissolved. Encapsulation via electrospinning using hydrophobic fibres is known (U.S. Pat. No. 9,096,845) and can be used for dry storage of bacteria.

Another method uses cross-linking of water-soluble polymers to form hydrogels and mixes them with a bacterial suspension (WO201006925). The bacteria-containing hydrogel particles are then electrospun together to form fibres (Gensheimer et al., Macromol. Biosci., (2011), 11 (3), 333-337). The method however requires multiple steps, each of which requires optimisation in order to have suitable stability in biorelevant aqueous conditions.

Co-axial electrospinning has also been used for the encapsulation of living cells into hydrophobic polymer fibres (Letnik et al., Biomacromolecules, (2015), 16(1), 3322-3328). Major disadvantages of co-axial electrospinning are the complexity of the setup and needle clogging problems.

The present inventors have found that the product of encapsulation via microfluidics can be directly electrospun to produce electrospun polymer fibres containing encapsulated bioactive entities. This means that encapsulation and electrospinning can be conducted consecutively as a single process using the same materials. A polymer solution in organic solvent is used as a continuous phase for the preparation of bioactive entity-containing microparticles in a microfluidic device. The resulting polymer solution comprising entrained microcapsules is directly electrospun into fibres.

### SUMMARY OF THE INVENTION

In a first aspect there is provided a method for producing electrospun polymer fibres comprising bioactive entity-containing microparticles, the method comprising the steps:
(a) encapsulation of a bioactive entity via microfluidics; and
(b) electrospinning into polymer fibres.

In particular, the encapsulation and electrospinning are performed using a single apparatus.

In particular the method may comprise encapsulation within a polymer (an encapsulation polymer, such as at least one hydrophilic and/or gel-forming polymer) in step a).

In a second aspect there is provided electrospun polymer fibres comprising bioactive-entity containing microparticles embedded within fibres of at least one hydrophobic polymer. The microparticles may also contain at least one hydrophilic and/or gel-forming polymer (encapsulation polymer).

The fibres and microparticles may correspond to any of the materials and methods discussed in any of the embodiments herein.

In a third aspect there is provided a wound dressing comprising electrospun polymer fibres wherein the fibres comprise bioactive-entity containing microparticles embedded within fibres of at least one hydrophobic polymer. Such fibres may be the fibres of the second aspect.

In a fourth aspect there is provided the use of electrospun polymer fibres, wherein the fibres comprise bioactive-entity containing microparticles embedded within fibres of at least one hydrophobic polymer, in enhancing wound healing and in the treatment of, acne, atopic dermatitis or scars. Such fibres may be the fibres of the second aspect.

In a fifth aspect there is provided an apparatus comprising a microfluidic device and an electrospinning device.

Various embodiments of the invention are described herein and are applicable to all aspects of the invention, where technically viable.

In one embodiment, the bioactive entity is a cell (e.g. bacterial cell) or active pharmaceutical ingredient (API).

In another embodiment, the bioactive entity comprises at least one type of bacteria, yeast, fungi and/or mammalian cell.

In another embodiment, the bioactive entity comprises bacteria, yeast, fungi and/or mammalian cells which produce a desired substance.

In another embodiment, the continuous phase comprises or consists of a hydrophobic polymer in organic solvent.

In another embodiment, the continuous phase comprises or consists of a copolymer of L-lactide and ε-Caprolactone (PLC) in dimethyl carbonate.

In another embodiment, the continuous phase comprises or consists of: a copolymer of L-lactide and ε-Caprolactone (PLC); poly(ethylene oxide) (PEO); and dimethyl carbonate.

### BRIEF DESCRIPTION OF THE FIGURES

FIG. 1 shows a schematic of an in situ microfluidic electrospinning set-up.

FIG. 2 shows scanning electron microscopy micrographs showing the morphology of PLC fibres with E. coli Nissle pSC101-Timer tet0-BFP prepared by coaxial electrospinning (A) and phase contrast (B) and confocal fluorescence (C) micrographs revealing the placement of bacteria.

FIG. 3 shows confocal fluorescence micrographs after encapsulation by in situ microfluidics electrospinning of E. coli MG1655 pC17_mCherry_lacI_tac_GFP (A) and E. coli labelled with mCherry (B, C, D) at varying places on the fibre mat.

FIG. 4 shows confocal fluorescence micrographs of E. coli BW25113 GFPmut2 bacteria (A), agarose microcapsules prepared with microfluidics loaded with the same bacteria (B) and those microcapsules after encapsulation by in situ microfluidics electrospinning (C).

FIG. 5 shows scanning electron microscopy (SEM) micrographs showing the morphology and fibre diameter size distributions of A. PLC fibres consisting of L. lactis IL1403 loaded agarose microcapsules and B. PLC/PEO fibres consisting of L. lactis IL1403 loaded agarose microcapsules prepared via in situ microfluidics electrospinning. C. Histograms of fibre diameter distributions.

FIG. 6 shows scanning electron microscopy micrographs showing the morphology of unloaded PLC fibres prepared by monoaxial electrospinning (A) in comparison with encapsulated E. coli nissle pSC101-Timer tet0-BFP-loaded PLC fibres (PLC fibres with microcapsules) prepared by in situ microfluidics electrospinning (B) and the comparative size distribution of the two fibre types (C)

FIG. 7 shows mechanical properties of PLC fibre matrices prepared by monoaxial electrospinning compared to encapsulated E. coli nissle pSC101-Timer tet0-BFP-loaded PLC fibre matrices (PLC fibres with microcapsules). Properties examined were Stress to breaking point (A), Young's Modulus (B), Elongation % at Break (C) and Tensile Strength (D).

FIG. 8 shows mechanical properties of PLC fibres, microcapsules loaded PLC fibres (consisting of E. coli nissle pSC101-Timer tet0-BFP), PLC/PEO fibres and microcapsules loaded PLC/PEO fibres (consisting of L. lactis IL1403). A. Hardness; B. Hardness work done; C. Deformation at hardness; and D. Thickness.

FIG. 9 shows swellability and degradation behaviour of PLC fibres prepared by monoaxial electrospinning compared to encapsulated E. coli nissle pSC101-Timer tet0-BFP-loaded PLC fibres (PLC fibres with microcapsules) in swelling index (A) and degradation over time (B).

FIG. 10 shows early degradation of PLC/PEO fibres and PLC/PEO fibres with microcapsules (consisting of L. lactis IL1403).

FIG. 11 shows safety of electrospun PLC+PEO fibre mats consisting of L. lactis bacteria-loaded agarose microcapsules. Cell viability tested on human primary skin fibroblasts (PF) and baby hamster kidney cells (BHK21) and MTS assay after 24 h (N=2). Two different media were used with and without antibiotics (penicillin and streptomycin). Cells on glass slides were used as controls. Keys: BHK—baby hamster kidney fibroblasts; PenStrep+—DMEM growth medium with penicillin and streptomycin; PF—primary human skin fibroblasts.

FIG. 12 shows electrospun fibres (12% PLC+0.3% PEO) consisting of agarose microcapsules with L. lactis (N=3) stained using dead-live stain Propidium iodine (PI) and SYTO-9.

FIG. 13A. Lactococcus lactis IL1403 grown on M17+lactate+0.5% glucose agar with pH indicator Bromocresol purple (Fisher scientific, Thermo Fischer, USA). Agar diffusion assay of L. lactis in minimally buffered M17 agar plates containing pH indicator bromocresol purple which turns yellow when pH drops below 5.2. The production of yellow colour around the bacterial colonies reveals the viability and can be used to assess the functionality of L. lactis which produce metabolite lactic acid as a result of their metabolism. B. Electrospun fibres (12% PLC+0.3% PEO) consisting of agarose microcapsules with living L. lactis (N=3) producing lactic acid into the surrounding environment. The effect is due to the reduced pH (lactic acid). The samples were kept at 30° C. for 24 h, 48 h and 72 h.

FIG. 14 shows green and red fluorescence intensity (A) and blue fluorescence intensity of E. coli nissle pSC101-Timer tet0-BFP within PLC fibres prepared during in situ microfluidics electrospinning (PLC fibres with microcapsules) (B) for different time-periods.

FIG. 15 shows leaching of E. coli nissle pSC101-Timer tet0-BFP from a PLC fibre mat after 24 h of incubation on LB agar plates (A) and the leaching of E. coli nissle pSC101-Timer tet0-BFP from PLC fibre mat after 48 h of incubation on LB agar plates where the fibre mat with folio was removed after 24 h of incubation (B) and (C). Bacterial growth indicates bacteria not held within the fibres. A small amount of bacterial growth is seen in each case.

FIG. 16 shows 15% PLC (Me₂CO3) and 0.625% agarose microcapsules consisting blue food dye (Blue Sky, UK) showing the dye release on the LB plate.

FIG. 17. A. Confocal microscopy images of L. lactis stained with FM-4-64 and +/−SYTO-9. B. Electrospun PLC (15% PLC in Me₂CO₃) or PLC/PEO (12% PLC+0.3% PEO in Me₂CO₃) fibres containing agarose microcapsules with living FM4-64 pre-stained L. lactis. SYTO-9 staining was added to the fibres after electrospinning. 488 nm laser was used for transmitted light images and for excitation of fluorophores. Emitted green fluorescence images were acquired in 493-558 nm range and red fluorescence images at 634-759 nm range.

FIG. 18A. CAM content within PLC and PLC/PEO fibres consisting of CAM-loaded agarose microcapsules. B. CAM release from electrospun PLC and PLC/PEO fibres consisting of CAM-loaded agarose microcapsules up to 4 h. C. CAM release from electrospun PLC and PLC/PEO fibres consisting of CAM-loaded agarose microcapsules up to 96 h.

FIG. 19 shows antibacterial activity of electrospun PLC fibres consisting of CAM-loaded agarose microcapsules and PLC+PEO fibres consisting of CAM-loaded agarose microcapsules together with respective control fibre mats without the drug against A. S. aureus and B. E. coli (N=3).

FIG. 20 shows antimicrobial peptide D-pleurocidin (D-Pleu) content within PLC and PLC/PEO fibres consisting of D-Pleu-loaded agarose microcapsules.

### DETAILED DESCRIPTION

The present invention provides a method for preparing electrospun polymer fibres loaded with microparticles comprising bioactive entities. The electrospun fibres are prepared via in situ microfluidic electrospinning.

The present inventors have surprisingly found that the encapsulated product from microfluidics can be directly electrospun, despite there being contradictory requirements for microfluidics and electrospinning. For example, polymer solutions used in electrospinning must be reasonably viscous, whereas in microfluidics the solutions cannot be too viscous.

Commonly after the microencapsulation process the particles are filtered, purified and dried. This applies particularly if the particles are to be used in electrospinning because the conditions for microencapsulation are rarely suitable for effective electrospinning. These separation steps require additional optimisation and time which may have a negative influence on the viability of bacteria or other bioactive entities within the microcapsules. This makes the process significantly more complex. In situ microfluidic electrospinning overcomes these challenges. In the present method, the polymer solution may be the continuous phase for encapsulation as well as an electrospinning solution. This approach enables direct electrospinning of encapsulated bioactive entities without any additional drying or purification steps. The protective polymer matrix surrounds the bioactive entity and these encapsulated bioactive entities are directly electrospun into fibres. The product is encapsulated bioactive entities within the fibres.

The present method also provides costs savings over existing methods due to easier scale up and reduced waste of the bioactive entity.

### Bioactive Entity

In some embodiments, the bioactive entity is a cell or active pharmaceutical ingredient (API).

In some embodiments, the cell is bacterium, yeast, fungus or mammalian cell, for example human cell.

In a particular embodiment, the cell is bacterium. In some cases, the bacteria are functional living bacteria.

In some cases, the cells (e.g. bacteria, yeast, fungi or mammalian cells, particularly bacteria) produce a desired substance. In some cases, the cells (e.g. bacteria) are genetically modified to produce the desired substance. In some embodiments, the cells (e.g. bacteria) produce at least one active substance naturally without genetic modification. One particularly preferred embodiment applicable to any aspect of the invention utilises bacterial cells which naturally produce at least one desired (e.g., active) substance (without artificial genetic modification).

In some cases, the desired substance is an antimicrobial substance, such as antimicrobial peptides and/or antibiotics. For example, nisin, colistin or defensins. In some cases, the desired substance is a skin structure/regeneration supporting compound such as allantoin, hyaluronic acid, salicylic acid, lactic acid, carotenoids, peptides (e.g. Thymosin Beta 4, (TB4)), proteins (e.g. collagen, elastin, laminin or fibrinogen), growth factors (e.g. PDGF, FGF, EFG or TGF-beta2), mitogens, morphogens, glycosaminoglycans, proteoglycans or combinations thereof.

The method of the present invention enhances the viability of bacteria during and after electrospinning. In some cases, 1 to 100% of the bacteria remain viable after encapsulation and electrospinning, such as at least 20%, at least 40%, at least 50%, at least 70% or at least 80%. In one embodiment, at least around 25% (e.g. 25% to 75%) of bacteria encapsulated within the electrospun fibres remain alive 48 hours after electrospinning. This is preferably at least 30% or at least 40%, more preferably at least 45%.

In some cases, there are 1.0E+04 bacteria per mm³ to 1.0E+06 bacteria per mm³, for example, 2.0E+05 bacteria per mm³.

In some cases, the bacteria are able to survive for at least 24 hours after electrospinning, preferably at least 48 hours, more preferably at least 72 hours.

The electrospun fibres allow two-way diffusion of substances through the pores on the fibres (e.g. nutrients in and produced substances out). In some cases, at least one growth medium is applied to the electrospun fibres. Suitable growth media include LB medium, LB agar or M9 with glucose.

In some embodiments, the API is a small molecule drug (e.g. ubidecarenone, epigallocatechin-3-gallate), peptide (e.g. antimicrobial peptide), protein (e.g. collagen, elastin, laminin or fibrinogen), nucleic acid, terpine (e.g. botulin), antibiotic (e.g. beta-lactam, aminoglycoside or macrolide antibiotic), antifungal agent, disinfectant, antimicrobial agent, growth factor, mitogen or morphogen.

### Microparticles

In some embodiments, the microparticles comprise at least one hydrophilic and/or gel-forming polymer. In particular, where the bioactive entity is at least one cell type, such as at least one bacterial cell, mammalian cell and/or fungal cell type, the microparticles may also comprise at least one hydrophilic and/or gel-forming polymer. Such encapsulation polymers are held within the microparticles along with the bioactive entity and form a part of the first input fluid to the microfluidic device utilised in various embodiments of the invention.

In some cases, the encapsulation polymer is a linear polymer.

In some embodiments, the encapsulation polymer may be a biopolymer, synthetic polymer or modified natural polymer. Such polymers may include polysaccharides, polyamino acids (e.g., peptides or proteins), polyesters, polyamides and/or polyethers.

In some cases, the encapsulation polymer is agarose, sodium alginate, gelatine, polyacrylamide, corn starch, polyethylene glycol (PEG), polyalkylene oxide (e.g. polyethylene oxide (PEO) or polypropylene oxide), polyacrylamide, poly(acrylic acid), polyvinylpyrrolidone, polyvinyl alcohol, N-(2-hydroxypropyl) methacrylamide (HMPA), polyphosphates, polyphosphazenes, water-soluble biopolymers such as xanthan gum, pectins, starch and its derivatives, cellulose ethers such as hydroxypropyl methylcellulose (HPMC), hydroxypropyl cellulose (HPC), hydroxyethyl cellulose (HEC), carboxymethyl cellulose (CMC) and its salts, hyaluronic acid and its salts, derivatives of chitosan, carrageenan dextran, guar gum, non-human albumin, gelatin, collagen, casein, soy protein (zein) or polymers forming hydrogels.

In a preferred embodiment, the encapsulation polymer is agarose.

In some embodiments, the microparticles are 20 to 300 μm in average diameter. In some cases, the microparticles are 20 to 100 μm, 50 to 100 μm or 50 to 80 μm in average diameter. These average diameters relate to the size of microparticles (also termed microcapsules) in isolation (e.g. in solvent) when the microparticles are substantially spherical. It is believed that when incorporated into microfibers the capsules become elongated within the fibres. In this case the aspect ratio of the capsules is much higher and the microcapsules may have a diameter in the smallest dimension which is similar to the diameter of the microfiber. It has also been observed that some “beads” of larger diameter form in the fibres. These beads may also contain microcapsules. It is believed that the microcapsules and entrapped bioactive entities are not limited to the “bead” sections of the fibres.

### Continuous Phase

The various aspects and embodiments of the present invention utilise a continuous phase. This typically comprises at least one polymer and at least one solvent. The continuous phase forms the second input fluid to the microfluidic device utilised in various embodiments of the invention. This fluid comprises at least one solvent and at least one electrospinning polymer and entrains microcapsules of bioactive entity (and optionally encapsulation polymer) as it flows through the microfluidic device.

In some embodiments, the electrospinning polymer comprises or consists of a biodegradable polymer.

In some embodiments, the electrospinning polymer comprises or consists of a hydrophobic polymer.

In some embodiments, the electrospinning polymer comprises or consists of a polyester, polyolefin, polyamide, polyether, polyketone, polyurethane, poly(ester urethane), polycarbonate, polyimide, polyacrylate, polysilsesquioxane, polyphosphazene, polysulfide or polysulfone, preferably a polyester.

In some embodiments, the electrospinning polymer comprises or consists of polyacrylonitrile, polyhydroxybutyrate, polystyrene, polyethylene, polypropylene, poly(ethylene oxide) (PEO), poly(vinyl alcohol); polylactide, polyglycolide, polybenzimidazole, polyethylene terephthalate, poly[ethylene-co-(vinyl acetate)], polyvinyl chloride, polymethyl methacrylate, polyvinyl butyral, polyvinylidene fluoride, poly(vinylidene fluoride-co-hexafluoropropylene), cellulose acetate, poly(vinyl acetate), poly(acrylic acid), poly(methacrylic acid), polyacrylamide, polyvinylpyrrolidone, poly(phenylene sulfide), hydroxypropyl cellulose, polyvinylidene chloride or polytetrafluoroethylene, polymethacrylate.

Preferably the electrospinning polymer comprises or consists of a homo- or co-polymer of lactic acid, glycolic acid and/or caprolactone. Most preferably the polymer comprises or consists of a copolymer of L-lactide and ε-Caprolactone (PLC). In some cases, the molar ratio of L-lactide to ε-Caprolactone is in the range of 80/20 to 20/80, preferably 70/30 to 30/70. Preferably the molar ratio of L-lactide to ε-Caprolactone is 70/30. In some cases the PLC has a molecular weight (MW) in the range 60,000 to 500,000 g/mol, preferably 150,000 to 250,000 g/mol.

In one embodiment the electrospinning polymer comprises or consists of at least one polyalkylene oxide such as polyethylene oxide (PEO) and/or polypropylene oxide (PPO). Typical MW of polyalkylene oxide (e.g. PEO) is in the range 20 kg/mol to 2000 kg/mol, such as 200 to 1500 kg/mol (e.g. 500 to 1200 kg/mol).

Preferably the electrospinning polymer comprises or consists of a mixture of PLC and PEO. It was surprisingly found that the combination of PLC and PEO increases the nanoporosity of the fibres. This may be the result of the water-soluble polymer dissolving out of the fibres following electrospinning and exposure to water. Water soluble polymers may dissolve in water at 25° C. to at least 20% (e.g. 20 to 99%) wt/vol. Water insoluble polymers may dissolve in water at 25° C. at a level of no more than 0.1% wt/vol (e.g. 0 to 0.1%), preferably no more than 0.01% wt/vol. In one embodiment, the electrospinning polymer comprises at least one water insoluble polymer such as a polyester (e.g. PLA, PLC etc), polyamide or polyolefin and at least one water-soluble polymer (such as a polyalkylene oxide (e.g. PEO, PPO), polyacid (e.g. polymalic acid), polyvinyl phosphate, polyvinyl sulphonate, polyvinyl acetate etc). The water-insoluble polymer and the water-soluble polymer may be present at a weight ratio of 80:20 to 99.9:0.1, preferably 10:1 to 99:1. In some embodiments, the electrospinning polymer comprises or consists of a biopolymer. In some embodiments, the electrospinning polymer comprises or consists of a blend of a biopolymer with a synthetic polymer. In some cases, the electrospinning polymer comprises or consists of collagen, gelatin, lipase, α-chymotrypsin, fibrinogen, silk, regenerated silk, regenerated Bombyx mori silk, silk fibroin, artificial spider silk, chitin, chitosan, cellulose or cellulose acetate. In some cases, the polymer comprises or consists of a blend of collagen and poly(ethylene oxide); collagen and poly(ε-caprolactone); collagen and polylactide-co-poly(ε-caprolactone); gelatin and poly(ε-caprolactone); gelatin and poly(ethylene oxide); casein and poly(vinyl alcohol); casein and poly(ethylene oxide); cellulose and poly(vinyl alcohol); bovine serum albumin and poly(vinyl alcohol); luciferase and poly(vinyl alcohol); Bombyx mori silk and poly(ethylene oxide); silk fibroin and chitosan; silk fibroin and chitin; silk and poly(ethylene oxide); chitosan and poly(ethylene oxide); chitosan and poly(vinyl alcohol); quaternized chitosan and poly(vinyl alcohol) or hexanoylchitosan and polylactide.

In some embodiments, the electrospinning polymer comprises or consists of a blend of two or more polymers, a copolymer or a blend of a polymer with an inorganic material. In some cases, the electrospinning polymer comprises or consists of a blend of polyvinylpyrrolidone and polylactide; polyaniline and polystyrene; polyaniline and poly(ethylene oxide); poly(vinyl chloride) and polyurethane; poly[(m-phenylene vinylene)-co-(2,5-dioctyloxy-p-phenylene vinylene)] and poly(ethylene oxide); a poly[2-methoxy-5-(2′-ethylhexyloxy)-1,4-phenylene vinylene] (MEH-PPV) and polystyrene; polyaniline and polystyrene; polyaniline and polycarbonate; poly(ethylene terephthalate) and poly(ethylene terephthalate)-co-poly(ethylene isophthalate); polysulfone and polyurethane; chitosan and polylactide, polyglycolide and chitin or polylactide and poly(lactide-co-glycolide). In some cases, the electrospinning polymer comprises or consists of a block copolymer. In some cases the block copolymer comprises or consists of a polylactide-b-poly(ethylene oxide) block copolymer; poly(lactide-co-glycolide)-b-poly(ethylene oxide) block copolymer; poly[(trimethylene carbonate)-b-(ε-caprolactone)] block copolymer; polystyrene-b-polydimethylsiloxane and polystyrene-b-polypropylene block copolymer; polystyrene-b-polybutadiene-b-polystyrene block copolymer or polystyrene-b-polyisoprene block copolymer.

In some embodiments, the solvent comprises or consists of an organic solvent.

In some embodiments, the solvent is immiscible or partially miscible with water.

In some embodiments, the solvent is miscible with water.

In some embodiments, the solvent may comprise or consist of at least one carbonate ester solvent such as a compound of formula CₙH₂ₙ₊₁O—CO—OCₘH₂ₘ₊₁. Where n and m are independently integers between 1 and 12, preferably between 1 and 6, such as 1, 2, 3 or 4. In one embodiment, n=m. In one embodiment, n=m=2 or preferably n=m=1.

In some embodiments the solvent is dimethyl carbonate, hexafluoroisopropanol, dimethylformamide, dimethylsulfoxide, acetonitrile, acetone, ethanol, hexamethylphosphoric triamide, N, N-diethylacetamine, N-methylpyrrolidinone, N-methylmorpholine-N-oxide monohydrate, ethyl acetate, chloroform, benzene, toluene, hexane, xylenes, cyclohexane, 1,1-dichloroethane, 1,2-dichloroethane, 1,1,1-trichloroethane, 1,1,2-trichloroethane, trichloroethane, carbon tetrachloride, dichloromethane, tetrachloromethane, diethyl ether, heptane, pentane, 2,2,4-trimethylpentane, diethyl carbonate, propyl lactate, butyl lactate or combinations thereof.

In a preferred embodiment, the solvent comprises or consists of a carbonate ester solvent. Preferably the solvent comprises or consists of dimethyl carbonate.

In one embodiment, the continuous phase is at least one biodegradable polyester in at least one carbonate ester solvent. In one embodiment, the continuous phase is at least one polyester of lactate, glycolate and/or caprolactone monomers in dimethyl carbonate (preferably PLC in dimethyl carbonate or a mixture of PLC and PEO in dimethyl carbonate). In some cases, the concentration of PLC in dimethyl carbonate is 11 to 20% (w/w), preferably 13 to 17% (w/w), more preferably 14 to 16% (w/w). In cases where the continuous phase comprises a mixture of PLC and PEO, the concentration of PLC in dimethyl carbonate is 10 to 20% (w/w), preferably 10 to 15% (w/w), more preferably 11 to 13% (w/w) and the concentration of PEO in dimethyl carbonate is 0.1 to 1.0% (w/w), preferably 0.1 to 0.5% (w/w), more preferably 0.1 to 0.3% (w/w). Fibres

In some embodiments, the electrospun polymer fibres have a mean diameter in the range of 0.5 to 50 μm, such as 1 to 10 μm, preferably 2 to 8 μm, more preferably 4 to 7 μm or 2 to 5 μm.

### Apparatus

Encapsulation and electrospinning are performed using a single apparatus. By a “single apparatus” is indicated that the two key steps of encapsulation and electrospinning are conducted concomitantly using (directly or indirectly) connected equipment. For example, the output of the encapsulation step may be used as the input for electrospinning step. Thus, the output of a microfluidic encapsulation device may be used as the input for an electrospinning device. Additional steps and/or devices such as solvent addition or removal steps, reservoirs for encapsulated material, monitoring and/or measuring steps etc may take place between the microfluidic encapsulation and electrospinning steps/devices. However, these two key operations take place together, preferably without any manual intervention (e.g. without manual transfer of material between devices). Thus, the devices required for microfluidic encapsulation and electrospinning operate together as a single apparatus.

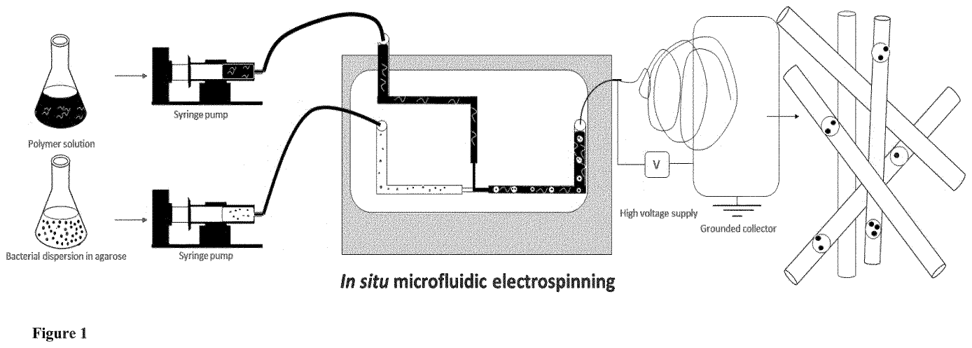

In some embodiments, the apparatus comprises, a microfluidic device and an electrospinning device (FIG. 1). In some cases, the microfluidic device is directly coupled to the electrospinning device. For example, the microfluidic device may be directly coupled to the electrospinning device by means of direct fluid flow from the output of the microfluidic device to the input of the electrospinning device. In some cases, the end channel of the microfluidic device leads to a fluid passage (e.g. a tube) which ends with the nozzle (e.g. needle) of the electrospinning device.

In some embodiments, the apparatus comprises a microfluidic device and an electrospinning device with fluid communication from the microfluidic device to the electrospinning device. The apparatus may also comprise at least one (preferably at least two) devices for inducing flow (e.g. for generating fluid pressure and/or fluid flow in at least one fluid within the apparatus). An example arrangement is illustrated in FIG. 1.

FIG. 1. Shows a schematic of an example in situ microfluidics electrospinning set-up. The microfluidic chip is shown enlarged with two input channels (polymer solution and bacteria in agarose) and one output channel with encapsulated bacteria entrained in polymer solution passing directly from the output channel to the electrospinning device.

The electrospinning device may comprise an outlet (e.g. a needle or other nozzle), a high-voltage power supply, and a grounded collection plate.

Additional components of the apparatus may include at least one of: a first reservoir of a first input liquid comprising bioactive entity in a suitable carrier (as discussed herein, e.g. of and API or bacterial, yeast, fungal and/or mammalian cells in a solution or suspension of encapsulation polymer such as agarose or API in solvent); a second reservoir of a second input liquid comprising continuous phase material (as discussed herein—e.g. of hydrophobic polymer in solvent solution); at least one device for generating a fluid pressure of the first input liquid (e.g. a pump or pressure reservoir); and/or at least one device for generating a fluid pressure of the second input liquid (e.g. a pump or pressure reservoir). Additional input liquids, optionally in reservoirs and optionally with corresponding devices for pressurising such liquids, may be present. Examples include additional solvents and/or additional active entities in suitable carrier liquids. Each input liquid will be introduced to the microfluidic device at one or more input channel of that device. Correspondingly, each reservoir may be in fluid communication with at least one input channel of the microfluidic device.

In some cases, the microfluidic device comprises at least two input channels and at least one output channel. Typically, the microfluidic device comprises more input channels than output channels (e.g. around two or more input channels for each output channel). For example, the microfluidic device may comprise two, three or four input channels and one or two output channels. One output channel is preferred. In a preferred configuration, the microfluidic device comprises two input channels and one output channel.

In some embodiments, the first input liquid is an aqueous solution comprising the bioactive entity (as discussed herein) and the second input liquid is a polymer solution in organic solvent (as discussed herein). In some embodiments, the microfluidic device (“chip”) is configured such that the at least two input liquids introduced in the at least two input channels meet within the device and generate a polymer solution comprising entrained microcapsules of bioactive entity (i.e. a continuous phase with entrained microcapsules of bioactive entity). Such continuous phase with entrained microcapsules of bioactive entity is conveyed by means of at least one channel of the microfluidic device to at least one electrospinning device and thus to the nozzle (e.g. needle) of the electrospinning device. In some embodiments, no purification or separation takes place between generating a continuous phase with entrained microcapsules of bioactive entity and reaching the nozzle of the electrospinning device.

The microfluidic device may be made of any suitable material, such as metal, glass, ceramic or polymeric material. In some embodiments, the microfluidic device is made from a polymer material such as an elastomeric polymer. In some cases, the microfluidic device is made from at least one silane polymer (e.g. polydimethylsiloxane (PDMS)) or at least one fluorinated polymer (e.g. polytetrafluoroethylene (PTFE)). Polydimethylsiloxane (PDMS) is a preferred material. Without being bound by theory, it is believed that use of a polymer material (e.g. an elastomeric polymer) such as a silane polymer and/or fluorinated polymer may reduce the tendency of the microfluidic device (“chip”) to clog or block in use.

In some embodiments, the microfluidic device (“chip”) is around 0.5 to 10 mm in each dimension (e.g. around 1 to around 5 mm in each dimension) and will preferably be no more than 2 mm in depth. Example sizes include 4×1×1 mm, 2×1×1 mm or 1×1×1 mm. Preferably, the microfluidic chip is 1×1×1 mm.

Suitable channels within the microfluidic device (chip) will be of any suitable cross-section, such as square, rectangular, semi-circular or semi-oval cross-section. Square or rectangular cross-sections (with either sharp or rounded bottom corners) are preferred. Suitable diameters for the channel cross-section may be around 0.01 to 1 mm, preferably 0.05 to 0.8 mm or 0.05 to 0.5 mm. In one embodiment, the microfluidic device may comprise channels with 0.4±0.1 mm cross-sections with a microcapsule forming section of 0.1±0.05 mm cross-section.

In general, any device which generates fluid pressure within at least one input fluid may be used as a device for inducing flow. Flow may also be induced by methods such as capillary action. In some embodiments, the device inducing flow is a pump. In some embodiments, the flow is induced by hydrostatic pressure (e.g. by use of pressurised reservoir or by gravitational pressure of fluid). One example of a suitable pump is a syringe pump.

In some embodiments, the flow rate for the first input liquid (e.g. a bioactive entity solution or dispersion) is 0.01-0.5 mL/h, such as 0.01-0.10 mL/h, preferably 0.02-0.09 mL/h, more preferably 0.025-0.08 mL/h. In some embodiments, the flow rate for the first input liquid (e.g. a bioactive entity solution or dispersion) is 0.01-0.30 mL/h, preferably 0.025-0.2 mL/h, more preferably 0.05-0.2 mL/h.

In some embodiments, the flow rate for the second input liquid (e.g. a polymer solution in solvent—as described herein) is 0.1-1.5 mL/h, such as 0.1-1.0 mL/h, preferably 0.2-0.7 mL/h, more preferably 0.4-0.5 mL/h. In some embodiments, the flow rate for the second input liquid (e.g. a polymer solution in solvent—as described herein) is 0.1-2.5 mL/h, preferably 0.8-1.5 mL/h, more preferably 0.4-0.8 mL/h.

In some embodiments, the rate of flow for the second input liquid (e.g. polymer solution) is 5 to 60 times greater than the rate of flow for the first input liquid (e.g. bioactive entity solution or dispersion). This may be, for example, 10 to 20 times greater.

In one embodiment, the electrospinning step is performed by coaxial or monoaxial electrospinning. Monoaxial electrospinning is preferred.

In some embodiments, the distance between the needle tip and collector plate is 10 to 15 cm, 12 to 15 cm, or 12 to 14 cm, preferably 13 cm.

In some embodiments, the voltage used for electrospinning is +/−3 to 60 kV, preferably +/−5 to 30 kV, preferably +/−10 to 17 kV, more preferably +/−10 to 15 kV.

In the description herein, a single flow path of at least two input channels of a microfluidic device to an output channel of that device, to an electrospinning device having at least one nozzle (e.g. needle) is generally described. Repetitions of such flow paths may, however, be made in parallel using a single or multiple microfluidic devices and a single or multiple electrospinning devices. Thus, the throughput of material may be increased by use of parallel flow paths which are, in effect, parallel repetitions of the flow path and apparatus described herein. In some embodiments, the throughput may be increased by 2 to 1000 (e.g. 2 to 100 or 2 to 20, such as 2, 5, 10, 100 or 1000) parallel flow paths, each beginning with at least two input channels of a microfluidic device and ending with at least one nozzle of an electrospinning device. In some embodiments, the throughput and efficiency are increased by using bifurcations of channels and parallel repetitions of nozzles.

### Method

In one embodiment, the hydrophilic and gel-forming polymer aqueous solution together with the bioactive entity is inserted into one microfluidic channel and polymer solution in organic solvent is inserted into the second microfluidic channel. The solutions mix together in the connection point between two inlet channels and one outlet channel and microparticles consisting bioactive entity coated with thin polymer layer are formed within a polymer solution.

In some embodiments, no filtration, drying or purification steps are required between steps a) and b).

In some embodiments, the bioactive entity not located in the fibres can be washed away after the in situ microfluidics electrospinning by immersing the fibre matrices into suitable washing medium (e.g. saline solution, phosphate buffered saline or Distilled water that may optionally comprise salts, antibiotics and/or cell culturing medium).

### Applications

Electrospun polymer fibres comprising bioactive entities (e.g. bacteria capable of producing antimicrobial substances) may be used for wound infection treatment and healing purposes. Where the bioactive entity is a cell type such as a bacterium, a sufficient encapsulation is required to shelter the cells in the wound environment and prolong the action time of the wound dressing. The fibres may be collected directly onto the wound. This provides the advantage that the encapsulated bacteria will immediately have access to food. This increases the viability of bacteria and their metabolic activity.

In some embodiments, the electrospun polymer fibres are used in wound healing (e.g. healing of epithelial wounds including wounds of the skin or mucosal surfaces such as oral wound healing). Examples include chronic non-healing ulcers, traumatic acute wounds, infected wounds and burns.

In some embodiments, the electrospun polymer fibres are used in treatment of infection, such as epithelial infection including infection of the skin or mucosal surface, such as infected wounds or burns to the skin or mucosa.

In some embodiments, the electrospun polymer fibres are used for the treatment of acne, atopic dermatitis, scars or dull skin, such as epidermolysis bullosa, skin burns, plastic surgery support or skin implants support.

### EMBODIMENTS

In addition to those embodiments indicated herein, which may be used individually or in any combination (where technically viable), the invention includes the following exemplary numbered embodiments:

1. A method for producing electrospun polymer fibres comprising bioactive entity-containing microparticles, the method comprising the steps:
(a) encapsulation of a bioactive entity via microfluidics; and
(b) electrospinning into polymer fibres
   2. The method of embodiment 1, wherein the bioactive entity is a cell or active pharmaceutical ingredient (API).
   3. The method of embodiment 1 or 2, wherein the bioactive entity comprises bacteria, yeast, fungi or mammalian cells.
   4. The method of embodiment 1 or 2 wherein the bioactive entity comprises an API, such as small molecule drugs, peptides or proteins.
   5. The method of any of embodiments 1 to 4, wherein encapsulation and electrospinning are performed using a single apparatus.
   6. The method of embodiment 5, wherein the apparatus comprises a microfluidic device and an electrospinning device.
   7. The method of any of embodiments 1 to 6, wherein the output of encapsulation step a) is the input of electrospinning step b).
   8. The method of any of embodiments 1 to 7, wherein the encapsulated bioactive entities are electrospun without any filtration, drying or purification steps between steps a) and b).
   9. The method of any of embodiments 1 to 8, wherein steps a) and b) are conduced consecutively using the same polymer(s) and solvent(s).
   9a The method of embodiment 9, wherein the continuous phase is or comprises a solution of at least one electrospinning polymer (which may be a polymer mixture) in at least one organic solvent.
   10. The method of embodiment 9, wherein the continuous phase is or comprises a hydrophobic polymer solution in organic solvent.
   11 The method of embodiment 10 wherein the hydrophobic polymer is a polyester, polyolefin or polysulfone.
   12. The method of embodiment 10 or 11, wherein the hydrophobic polymer is a homo- or co-polymer of lactic acid, glycolic acid and/or caprolactone.
   13. The method of any of embodiments 10 to 12, wherein the electrospinning (e.g. hydrophobic polymer) comprises or consists of a copolymer of L-lactide and ε-Caprolactone (PLC).
   13a The method of any of embodiments 10 to 12, wherein the electrospinning polymer comprises or consists of a mixture of at least one water-insoluble (e.g. hydrophobic) polymer and at least one water-soluble polymer.
   13b The method of any of embodiments 10 to 12, wherein the electrospinning polymer comprises or consists of a mixture of PLC and PEO.
   14. The method of embodiment 13 or 13a, wherein the organic solvent comprises or consists of dimethyl carbonate.
   15. The method of embodiment 14, wherein the continuous phase is PLC in dimethyl carbonate.
   15a. The method of embodiment 13, wherein the continuous phase is PLC and PEO in dimethyl carbonate.
   16. The method of embodiment 15, wherein the concentration of PLC in dimethyl carbonate is 11 to 20% (w/w), preferably 15% (w/w).
   16a. The method of embodiment 14a, wherein the concentration of PLC in dimethyl carbonate is 10 to 20% (w/w), preferably 12% (w/w) and the concentration of PEO in dimethyl carbonate is 0.1 to 1.0% (w/w), preferably 0.3% (w/w).
   17 The method of any of embodiments 1 to 16, wherein the bioactive entities are encapsulated in hydrophilic and/or gel-forming polymer microparticles.
   18. The method of embodiment 17, wherein the hydrophilic and/or gel-forming polymer is agarose.
   19 The method of any of embodiments 1 to 18, wherein in step a) the bioactive entities are encapsulated in microcapsules of diameter 50 to 100 μm (when measured outside of the fibres).
   20. The method of any of embodiments 1 to 19, wherein the voltage used for electrospinning is +/−3 to 60 kV.
   21. The method of any of embodiments 1 to 20, further comprising:
(c) washing the polymer fibres to remove bioactive entity not located within the fibres.
   22. The method of any of embodiments 1 to 3 and 5 to 21, wherein the bioactive entity comprises bacteria.
   23. The method of embodiment 22, wherein 1-100% of the bacteria remain viable after encapsulation and electrospinning.
   24. The method of embodiments 22 or 23, wherein the bacteria are functional living bacteria.
   25 The method of any of embodiments 22 to 24, wherein the bacteria produce a desired substance.
   26. The method of embodiment 25 wherein the desired substance is an antimicrobial substance, such as antimicrobial peptides and/or antibiotics.
   27 The method of embodiment 25, wherein the desired substance is a skin structure/regeneration supporting compound, such as allantoin, hyaluronic acid, salicylic acid, lactic acid, carotenoids, peptides, proteins, growth factors, mitogens, morphogens, glycosaminoglycans and/or proteoglycans.
   28. The method of any of embodiments 1 to 27, wherein the electrospun polymer fibres are used for wound healing and/or infection treatment application.
   29. The method of any of embodiments 1 to 27, wherein the electrospun polymer fibres are used for the treatment of acne, atopic dermatitis or scars.
   30. Electrospun polymer fibres comprising bioactive entity-containing hydrophilic and gel-forming polymer microparticles embedded within fibres of at least one hydrophobic polymer.
   31. The electrospun polymer fibres of embodiment 30 formed or formable according to the method of any of embodiments 1 to 29.
   32. The electrospun polymer fibres of embodiment 30 or embodiment 31, wherein the electrospun polymer fibre comprises PLC.
   32a. The electrospun polymer fibres of embodiment 30 or embodiment 31, wherein the electrospun polymer fibres comprises PLC and PEO.
   33. The electrospun polymer fibres of any of embodiments 30 to 32, wherein the bioactive entity comprises bacteria.
   34 The electrospun polymer fibres of embodiment 33 containing at least 1.0E+05 viable bacteria per mm³.
   35 The electrospun polymer fibres according to embodiment 33 or 34, wherein the bacteria are capable of producing at least one desired substance, such as an antimicrobial substance.
   36. The electrospun polymer fibres according to any of embodiments 30 to 35 for use in wound healing and/or infection treatment.
   37. The electrospun polymer fibres according to any of embodiments 30 to 35 for use in the treatment, prevention or reduction of acne, atopic dermatitis or scars.
   38. A wound dressing comprising electrospun polymer fibres of any of embodiments 30 to 35.
   39. Use of electrospun polymer fibres as embodimented in any of embodiments 30 to 35 in enhancing wound healing.
   40 An apparatus comprising a microfluidic device and an electrospinning device.
   41 The apparatus according to embodiment 40 wherein the output of the microfluidic device is connected directly to the electrospinning device without any filtering or drying device between.
   42 The apparatus according to embodiment 40 comprising at least two devices for inducing flow, a microfluidic device, a nozzle, a high-voltage power supply, a grounded collection plate and an electrospinning device, wherein the microfluidic device comprises at least two input channels and at least one output channel, wherein the first liquid is an aqueous solution containing bioactive entity, the second input liquid is a polymer solution in organic solvent, the microfluidic device is configured such that the two input liquids introduced to dedicated input channels, meet and generate a polymer solution comprising entrained microcapsules of bioactive entity which pass to the nozzle, and the nozzle is held at a potential of +3-60 kV or −3-60 kV relative to the grounded collection plate.
   42. The apparatus of embodiment 41, wherein the second device inducing flow is configured to deliver the polymer solution at a rate 5-60 times greater than the rate of delivery of the aqueous solution by the first device inducing flow.
   43. The apparatus of any of embodiments 40 to 42, wherein the microfluidic device is made of polydimethylsiloxane (PDMS).

### EXAMPLES

### Materials

Copolymer of L-lactide and &-Caprolactone (PLC) in a 70/30 molar ratio (Purasorb PLC7015, Purac Corbion, The Netherlands, inherent viscosity midpoint of 1.5 dL/g, MW 202 000 g/mol; chemical name: (3S-cis)-3,6-dimethyl-1,4-dioxane-2,5-dione, polymer with 2-oxepanone).

Poly(ethylene oxide) (PEO), SENTRY™ POLYOX™ WSR 1105-LEO NF Grade, DOW Chemicals, MW 900 000 g/mol; chemical name: α-hydro-ω-hydroxypoly(oxyethylene).

E. coli MG1655, E. coli BW25113 and probiotic bacterium E. coli Nissle 1917 (Mutaflor, Pharma-Zentrale GmbH, Germany). E. coli MG1655 pC17_mCherry_lacI_tac_GFP (fluorescent GFP and mCherry), E. coli BW25113 GFPmut2 (fluorescent GFP), E. coli nissle pSC101-Timer tet0-BFP (fluorescent Timer and BFP). Genetically modified bacteria (GMO) were used to visualise the bacteria.

Probiotic Gram-positive bacterium L. lactis IL1403 (TF-TAK, Estonia).

The pathogenic bacteria used in assessing the antibacterial activity of electrospun drug-loaded matrices were purchased from the Leibniz Institute DSMZ-German Collection of Microorganisms and Cell Cultures (Braunschweig, Germany): E. coli (DSM no. 1103 (clinical isolate) and S. aureus DSM 2569 (clinical isolate).

Bacteria were stained using red-fluorescent membrane stain FM-4-64, green-fluorescent nucleic acid stain SYTO-9 and red-fluorescent nucleic acid stain propidium iodine, PI (all stains purchased from Invitrogen, Thermo Fischer, USA).

Antibacterial agent chloramphenicol (PubChem CID: 5959; Sigma-Aldrich) and antimicrobial peptide D-Pleurocidin (Cambridge Research Biomedicals (Cleveland, UK) as desalted grade (crude) and purified using reverse phase chromatography) were used for the preparation of drug delivery systems (DDSs) using in situ droplet microfluidics electrospinning.

### Methods

### Microfluidics

A chip made of polydimethylsiloxane (PDMS) was prepared. The three-channel (two inputs and one output) connection was built of channels of a square-section, with overall channel diameter of 0.40 mm, and a microcapsule forming section diameter of 0.1 mm. The microfluidic chip was 1×1×1 mm. Different flow conditions were tested from 0.01 mL/h-1.5 mL/h and the most suitable one was selected for both channels. When a single polymer solution was used, flow conditions were: 0.4-0.5 mL/h for the polymer solution and 0.05-0.08 mL/h for the bacterial dispersion or 0.4-0.8 mL/h for the polymer solution and 0.05-0.08 mL/h for the bacterial dispersion. When two polymer solutions were used, flow conditions were: 0.8-1.5 mL/h for the two polymer solutions (12% PLC+0.3% PEO) and 0.05-0.2 mL/h for the bacterial dispersion.

### Preparation of Electrospinning Solutions

For the preparation of electrospinning solutions, PLC solutions in dimethyl carbonate in different concentrations and/or PLC solutions with PEO in dimethyl carbonate in different concentrations were prepared. All polymer solutions were kept on a magnetic stirrer for one-day prior use. PLC solutions with PEO needed heating during dissolving, therefore the solutions were heated on the heat-plate at 40° C. with constant stirring for 24 h prior to electrospinning. Before electrospinning the solutions were allowed to cool down to room temperature. For the preparation of microcapsules, agarose aqueous solution in different concentrations were prepared. Solutions were prepared in vials and left on heated (90° C.±2° C. or 45° C.±2° C.) magnetic stirrer overnight.

Drug-loaded electrospinning solutions were prepared for the testing of the in-situ microfluidics electrospinning method to produce drug delivery systems (DDSs). PLC, PLC+PEO solutions and agarose solutions were prepared as for the preparation of bacteria loaded electrospun fibres, but instead of bacteria, antibacterial agents chloramphenicol (CAM) and/or antimicrobial peptide (D-Pleurocidin) were added to the agarose solution. CAM (0.025 g) was added while preparing 0.625% agarose aqueous solution (ad 10 g) and stirred on a magnetic stirrer at 45° C. overnight prior to electrospinning. AMP was added immediately prior to electrospinning to avoid possible degradation during stirring overnight and/or heating during agarose solution preparation. D-Pleurocidin was weighed (0.01528 g), ready agarose solution added (4.87 g) and obtained solution stirred for 30 minutes prior to electrospinning.

### Viscosity of Electrospinning Solutions

The viscosities of electrospinning solutions were measured with DVNext Cone&Plate rheometer (Brookfield, USA) using CP-52 spindle at 5 RPM (at ambient environmental conditions; temperature protocolled). The volume of the sample was 0.5 mL.

### Electrospinning

Electrospinning was performed using an ESR200RD robotized electrospinning system using monoaxial set-up. Electrospinning process parameters were varied to achieve homogenous and reproducible fibre formation. For microencapsulation together with in situ electrospinning, microcapsules were formed in microfluidics and directly electrospun into fibres (FIG. 1). The set-up consisted of two inlet channels and one outlet channel. In order to produce homogenous microcapsules, the two syringe pumps work at different speeds and the speed ranges vary for different formulations, although the ratio between the two channels flow rates is similar. For single polymer (PLC) the flow rates are 0.4-0.5 mL/h for the polymer solution and 0.05-0.08 mL/h for the bacterial dispersion or 0.4-0.8 mL/h for the polymer solution and 0.05-0.08 mL/h for the bacterial dispersion. For two polymer mixture solution (PLC+PEO) the flow rates are 0.8-1.5 mL/h for the polymer solution and 0.05-0.2 mL/h for the bacterial dispersion. The microcapsules form in the T junction of the microfluidic device and exit from the outlet channel towards the needle attached to the exit tube. The electrospinning electrode is attached to the needle and the fibres are collected on either a glass microscope slide (for microscopy images) or aluminum foil or solid LB plate.

Total of 30-100 μL was electrospun either on a metal collector plate covered with aluminum folio (SEM imaging, mechanical properties, contact angle, degradation, drug content and release) or on glass slides for microscopy analyses (glass size: 24×24 mm) and for agar diffusion assays and safety assays (circle glass slides with a diameter of 12 mm). Plastic syringes (3 mL) were used and single use blunt needle B. Braun of 21G (inner diameter of 0.51 mm) was used. Electrospinning parameters were voltage of 13-16.5 kV and flow rate was varied for different channels from 1.5 mL/h to 0.05 mL/h and distance between the needle and collector 130 mm. Humidity varied between 19 and 50% and temperature between 23.8° C. to 33.0° C. To keep the RH in the electrospinning chamber in specified ranges (19-30%) dehumidifier (COTES A/S, Denmark) was used. The fibres were collected on a piece of aluminum foil and/or onto the Petri dishes consisting of LB medium or 1.5% LB agar. Ready samples on foil were put into Ziploc bags. The samples were kept at room temperature (21±2° C.) in a desiccator at 0% relative humidity (RH) above silica gel to avoid humidity induced changes in the mats. Fibre samples collected on microscopy slide were immediately put on top of agar pads (1% agarose with M17-glucose media) and either visualized under microscopy or stored at 30° C. or in fridge (+4° C.) depending on the assay of interest. Fibre samples collected on circled glass microscopy slides were immediately put on top of agarose plates (1% agarose with M17-glucose media with or without indicator bromocresol purple) and kept at 30° C. for specified time periods before analysis (scanning/colored zone measurements).

Drug-loaded fibres were electrospun using the same microfluidic chip and electrospinning equipment as for bacteria-loaded fibres preparation and the following conditions-nozzle to collector distance: 9.5-11 cm, voltage (U): 13-16.5 kV, temperature (T): 24-38.7° C., relative humidity (RH) 8-42%. Flow rates of the solutions depended on the exact formulation: for CAM-PLC15% formulation continuous phase (polymer solution) flow rate was 0.5-0.8 mL/h, and dispersed phase 0.05-0.2 mL/h. Weight of the CAM-loaded fibre mat produced was 0.13-0.16 g. For PLC 12%+PEO 0.3% formulation (both CAM-loaded and D-Pleurocidin-loaded) the conditions were: distance 11 cm, U-16.1 KV, T: 31.0-38.7° C., relative humidity (RH) 31.1-42.0%. The flow rates for PLC 12%/PEO 0.3% were 0.8-1.2 mL/h (for polymer solution and 0.1-0.3 mL/h for dispersed phase). Weight of the CAM-loaded fibre mat produced was 0.15-0.16 g, and 0.31 g for D-Pleurocidin loaded mat.

Droplet formation was monitored by a camera. An optic microscope was used to observe fibres before the experiment and drug-loaded mat of electrospun micro- and nanofibers was collected on a non-sticking aluminum foil for 1 h up to 3 h. For the antibacterial activity agar diffusion assay the CAM-loaded fibre mats were collected directly on the small glass slides (diameter of 12 mm) by electrospinning for 90 min.

### Coaxial Electrospinning (Comparative)

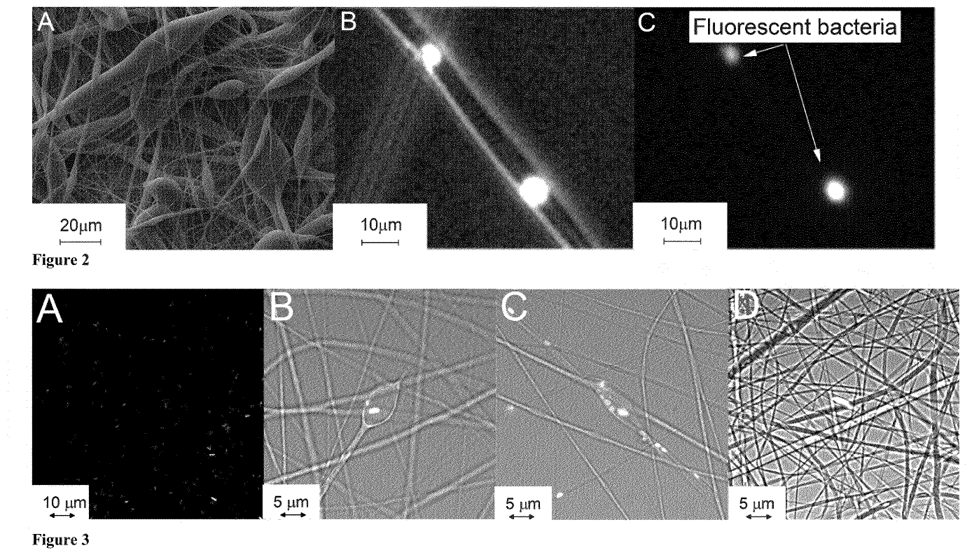

In order to test the possibility of using some other approach for obtaining the bacteria-loaded matrices, coaxial electrospinning was used together with the same materials (PLC polymer, dimethyl carbonate solvent and E. coli bacteria). However, it was not easy to use coaxial electrospinning with the same materials. The electrospinning process was not stable and beaded fibres were obtained (FIG. 2A). The confocal imaging analysis showed that much less bacteria were encapsulated in the fibres compared to the fibres prepared by in situ microfluidics electrospinning (FIGS. 2B and 2C).

### Viability and Presence of Bacteria

For bacteria-loaded fibre production, overnight grown bacterial dispersion was centrifuged and taken up in an agarose solution in order to obtain a specified bacterial concentration (OD at 600 nm 2.0-4.5) and used for in situ microfluidics electrospinning.

Bacterial distribution within hydrophobic fibres (PLC fibres) was evaluated with scanning electron microscopy (SEM). To evaluate the viability and metabolic activity of bacteria the expression of bacterial reporter-protein TIMER was used. The green and red fluorescence was measured using ImageJ MicrobeJ plugin (National Institutes of Health, Bethesda, MD, USA) from confocal images of the samples and intensity was calculated. To further evaluate the metabolic activity, blue fluorescent protein (BFP) was induced with doxycycline (DOX) 4 h prior measurement and analysed under confocal microscope. The blue fluorescence intensity was measured using ImageJ MicrobeJ plugin. The viability and metabolic activity of bacteria within growth medium (M9) were monitored for different time-periods (24 h, 48 h, 72 h).

### Encapsulation Efficiency of Bacteria and Purity of the Fibre Mats

The fibre mats were removed from folio and put onto LB plates. Loose bacteria outside the fibres diffuse into the LB agar and start growing there after 24 h at 37° C. In addition, for the visualization of the bacteria within and between electrospun fibres confocal microscopy was used. Bacteria were stained prior electrospinning with membrane stain FM4-64 (Invitrogen).

Dead-Live Staining Assay with Propidium Iodine

The viability and functionality of L. lactis 1403 strain bacteria within the electrospun fibres (as well as to prove the diffusion of substances into and out of the fibres) were investigated using the dead-live staining propidium iodine, PI and SYTO9 and confocal microscopy. After electrospinning the fibre mats were put on the M17+glucose agarose pads and stored at 4° C. until imaging was performed, usually the next day. For staining 20 μL of SYTO9 stock 500× dilution in H2O and 20 μL of PI stock 500× dilution in H2O were added to the agarose pad (opposite side from the fibres and diffusion of dyes was allowed to take place at least 20 min before imaging). A bacterial mixture consisting of live (overnight liquid bacterial culture) and dead L. lactis (heat treated at 56° C. for 10 min) bacteria in 50:50 ratio was used as a control for imaging. Images were collected with 488 nm laser excitation and emission in green and red channels. All cells were SYTO9 positive. If PI has entered the cells SYTO-9 fluorescence gets quenched, and signal is lower. Overlay image discriminated well between live (green) and dead (red) bacteria.

### Bacterial Metabolite Lactic Acid Production on Agar Plate (pH Reduction Assay)

The viability and functionality of L. lactis bacteria within the fibres were measured by the production of bacterial metabolite lactic acid. The latter was measured when Lactococcus lactis 1403 strain was grown on agarose plates with glucose (as described in the literature DOI: 10.1126/scitranslmed.aao258). Briefly, the M17 1% agarose plates with glucose (final concentration of 0.5%) were made together with pH indicator bromocresol purple (Fisher scientific, Thermo Fischer, USA) (4.6 mg of indicator in 200 mL medium) which changes its colour in different pH conditions (from purple to yellow in acidic environment). Electrospun small glass slides were put onto these agarose plates and samples were kept at 30° C. for 24 h, 48 h and 72 h. At each time point images were collected by scanner (Epson, Japan). Colour change zones were measured from these images.

### Characterisation of Electrospun Fibres

Fibre morphology was studied using optical microscopy (CETI MagtexT, UK; 40× magnification). More depth morphology and size analyses were performed using SEM (Zeiss EVO® 15 MA, Germany). Samples were sputter coated using approximately 3 nm thick platinum layer. Confocal fluorescence microscopy (CFM) (LSM710, Carl Zeiss, Germany) was used to visualise the labelled and/or stained bacteria within fibres. The presence and distribution of bacteria within hydrophobic fibres (PLC fibres/PLC+PEO fibres) were evaluated with scanning electron microscopy (SEM) and confocal fluorescent microscopy (with bacterial staining (FM 4-64 and SYTO9). Zen software was used for the analysis (Zeiss). SEM was also used for morphology and diameter analysis of all electrospun fibres (pristine and bacteria-loaded).

### Contact Angle

In order to understand the hydrophilic/hydrophobic nature of the fibre scaffolds and their wettability behaviour, the contact angle between the fibre scaffolds (m=11.5±0.01 mg; size 2×2 cm) and distilled water was measured with the sessile drop method (OCA 15EC, DataPhysics Instruments GmbH, Filderstadt, Germany). A drop of distilled water (5 μl) was applied onto the fibre scaffolds. The contact angle measurements were taken at time-point 0 and 30 s after the liquid drop touched the surface of the fibe scaffold. This test was carried out at RT (22-31.0° C.±0.3). The contact angle was analyzed using the SCA20 software (DataPhysics Instruments GmbH, Filderstadt, Germany). Each sample was measured at least in triplicate.

### Swellability and Degradation Analysis

For swelling index and weight loss measurements (early degradation) a set of 4 cm² square-shape samples (N=3) were cut from the fibre scaffolds and weighed, then immersed into 10 mL of 1×PBS solution and kept statically at 37° C. for 24 h. Static conditions were selected in order to mimic the wound conditions. The samples were then removed from 1×PBS and washed with distilled water and placed on a plastic Falcon Cell strainer sieves (mesh size 40 μm, 70 μm diameter) (Fisher scientific, Thermo Fischer, USA) placed on a 50 mL Falcon tube to remove free surface solution and weighed. Wet samples were lyophilised for 24 h. After 24 h the dry weight of the samples was measured and SEM images taken from the samples. Swelling index and weight loss were calculated as reported previously (Nazemi et al., BioMed Research International, (2014). Prolonged degradation measurements were performed at different time-points by measuring the mass loss for up to 1 week. Triplicate samples (thickness of approximately 50 μm for the PLC fibre mat and 60 μm for the microcapsule loaded fibre mat thickness of approximately 60 μm for the PLC/PEO fibre mat and 87 μm for the microcapsule loaded PLC/PEO fibre mat) were incubated in 1 mL 1×PBS statically at 37° C. At each time point, fibre mats were removed from the buffer, rinsed in deionized water, dried via lyophilisation for 24 hours, and weighed. The medium was not changed until the targeted time point to minimise phase separation errors resulting from disintegration of the mat at longer time points. The fibre mat morphology was assessed by SEM.

### Mechanical Analyses

Mechanical behavior of the bacteria loaded and unloaded fibre mats was studied by Brookfield CT3 Texture Analyzer (Middleboro, MA, USA) equipped with a 10 kg load cell. Puncture test was performed using TexturePro CT software (AMTEK Brookfield, Middleboro, MA, USA). 2×2 cm pieces were used for analysis which were secured between the film support fixture (TA-FSF) and punctures were made with a cylinder probe (TA-42, diameter 3 mm). The target distance 40 mm was used with all the samples with trigger load 5 g and test speed 2.5 mm/s. All measurements were performed at ambient conditions (temperature of 22±1° C. and RH of 20±2%). Each sample group comprised at least 3-5 specimens. The mean thickness of the fibre mats varied from 0.05-0.1 mm measured using Precision-Micrometer 533.501 (Scalamesszeuge, Dettingen, Germany) with a resolution of 0.01 mm. Young's modulus (MPa, linear region) and tensile strength (zero slope) were calculated from each corresponding stress-strain curve. The same TexturePro CT software was used to obtain the elongation at break (%) values. The applied force (N) and distance of the probe (mm) were recorded as the probe deformed the sample and hardness (N), deformation at hardness (mm) and hardness work done (mJ) were calculated.

### Food Dye Diffusion Assay

Preliminary testing of the diffusion of substances out from the electrospun fibres was measured using Blue food dye (Sky blue, UK). The samples were prepared using the same 15% PLC+agarose formulation, but instead of bacteria, food dye was added into the agarose solution. Firstly, the fibre matrices were put onto LB plates and the diffusion of dye was monitored. Secondly, the diffusion of dye into the buffer solution was evaluated by immersion of samples into phosphate buffer solution (pH 7.4) and the absorbance was measured using microtiter plate reader at two different wavelengths (410 and 630 nm).

### SYTO9 Diffusion Assay

The transport of substances into the electrospun fibres consisting of microcapsules was proven using microscopy and nucleic acid stain SYTO9. Briefly, collected 12% PLC/PEO fibre mats consisting of L. lactis loaded agarose microcapsules on microscopy cover glass were put onto the 1% agarose pads consisting of M17-glucose media, covered with another cover glass and analyzed immediately or incubated at 30° C. for specified time periods (24 h, 48 h or 72 h). These samples were photographed with the confocal fluorescent microscopy before and after addition of SYTO9 stain (20 μL of 50 μM stain per sample).

Drug Content and Release from Fibres

The drug content and released drug amounts were measured by HPLC. Weighed CAM-loaded fibre mats (0.03-0.04 g; n=4) were dissolved in chloroform:methanol 3:1 v/v (0.5 mL), precipitated with pure methanol (2.4 mL), filtered through a syringe filter (filtration through a Whatman 0.45 μm TF filter), and dispatched for drug content analysis. Weighed D-Pleurocidin-loaded fibre mats (0.07-0.17 g; n=3) were dissolved in 1,1,1,3,3,3-hexafluoroisopropanol (HFIP, 99.5%; Sigma-Aldrich) (3 mL), filtered through a syringe filter (filtration through a Whatman 0.22 μm Nylon filter), and dispatched for drug content analysis.

The in vitro drug release of CAM from drug-loaded electrospun fibre mats was carried out using 0.12-0.15 g of fibre samples (n=4). These were weighed, placed into 10 mL of PBS (pH 7.4) at 37° C. in 50 mL plastic tubes. The tubes were put into dissolution apparatus vessel (dissolution system 2100, Distek Inc., NJ, USA) containing water and maintained at 37° C. The tubes were rotated by the paddles at the speed of 100 rpm. Aliquots of 200 μL were removed and replaced with the same amount of 1×PBS buffer at set time points. Samples were collected after specified timepoints 0 min, 5 min, 4 h, 24 h and 1 week. The aliquots were analyzed using HPLC. Control samples consisted of 1 mg/mL and 0.005 mg/mL CAM solutions in 1×PBS.

### Antibacterial Activity of Drug Loaded Electrospun Fibres

Agar diffusion assay using overnight liquid cultures of wound pathogens E. coli and S. aureus taken from single colony on agar plates and the cell number was adjusted with fresh LB to about 3×10E7 colony-forming units (CFU)/mL. 100 μL of these dilutions were spread onto the surface of LB agar plates. Glass discs covered with electrospun CAM-loaded fibre matrices and control PLC and PLC/PEO fibre matrices with a diameter of 12 mm were applied to these plates. Positive controls were prepared by immersing 6 mm filter paper discs with 5, 10 and 20 μL of CAM solution. Untreated filter paper was used as a negative control. The plates were incubated at 37° C. for 24 h. The inhibition zones free of bacterial growth were determined. Tests were run in triplicate.

### Safety of Electrospun Bacteria-Loaded Fibre Mats

Safety of the L. lactis bacteria-loaded fibre matrices was tested by measuring the cell viability of primary skin fibroblasts and baby hamster kidney (BHK-21) fibroblasts on fibre mats in biorelevant conditions using MTS assay. MTS activity was measured, and this activity was related to the metabolic activity and cell viability in the presence of electrospun fibre matrices. Electrospun fibres collected on glass slides (diameter of 12 mm) were put into the 24-well plate wells. Cells at the density of 50.000 cells in 500 μL DMEM (with and without penicillin+streptomycin) per well were seeded on the fibre mats. Additional 750 μL of media was inserted into the wells and incubated together with cells at 37° C. and 5% CO₂ for 24 h. As controls, untreated cells grown on the glass slides and bacteria-loaded fibre mats without any eukaryotic cells were used. Background wells were created adding 1.25 mL of fresh medium into the empty wells. After incubation, fibre mats on slides with cells were removed and washed twice using 1×PBS buffer. Fibre mats on slides were placed into 24-well plates and 500 μL Dulbecco's modified eagle's medium/nutrient mixture f-12 ham (Sigma, Germany) was added. Control glass slides with cells were also washed twice with 1×PBS and medium was changed. 50 μL of MTS Cell Proliferation reagent was added into each well. Additional 1 h of incubation in the same conditions was carried out, until colour change was visible. 200 μL samples from the 24-well plate were transferred to the 96-well plate, making technical duplicates for each. Then absorbance was measured using a microplate reader at OD=490 nm.

### Expression of Bacterial Reporter-Protein TIMER.

To evaluate the viability and metabolic activity of bacteria (E. coli) the expression of bacterial reporter-protein TIMER was used. The green and red fluorescence was measured using ImageJ MicrobeJ plugin (National Institutes of Health, Bethesda, MD, USA) from confocal images of the samples and intensity was calculated. To further evaluate the metabolic activity blue fluorescent protein (BFP) was induced with doxycycline (DOX) 4 h prior measurement and analysed under confocal microscope. The blue fluorescence intensity was measured using ImageJ MicrobeJ plugin. The viability and metabolic activity of bacteria within growth medium (M9) were monitored for different time-periods (24 h, 48 h, 72 h).

Dead-Live Staining Assay with Propidium Iodine.

The viability and functionality of L. lactis 1403 strain bacteria within the electrospun fibres (as well as to prove the diffusion of substances into and out of the fibres) were investigated using the dead-live staining propidium iodine, PI and SYTO9 and confocal microscopy. After electrospinning the fibre mats were put on the M17+glucose agarose pads and stored at 4° C. until imaging was performed, usually the next day. For staining 20 μL of SYTO9 stock 500× dilution in H2O and 20 μL of PI stock 500× dilution in H2O were added to the agarose pad (opposite side from the fibres and diffusion of dyes was allowed to take place at least 20 min before imaging). A bacterial mixture consisting of live (overnight liquid bacterial culture) and dead L. lactis (heat treated at 56° C. for 10 min) bacteria in 50:50 ratio was used as a control for imaging. Images were collected with 488 nm laser excitation and emission in green and red channels. All cells were SYTO9 positive. If PI has entered the cells SYTO-9 fluorescence gets quenched, and signal is lower. Overlay image discriminated well between live (green) and dead (red) bacteria.

### Bacterial Metabolite Lactic Acid Production on Agar Plate (pH Reduction Assay).

The viability and functionality of L. lactis bacteria within the fibres were measured by the production of bacterial metabolite lactic acid. The latter was measured when Lactococcus lactis 1403 strain was grown on agarose plates with glucose (as described the literature DOI: in 10.1126/scitranslmed.aao258). Briefly, the M17 1% agarose plates with glucose (final concentration of 0.5%) were made together with pH indicator bromocresol purple (Fisher scientific, Thermo Fischer, USA) (4.6 mg of indicator in 200 mL medium) which changes its colour in different pH conditions (from purple to yellow in acidic environment). Electrospun small glass slides were put onto these agarose plates and samples were kept at 30° C. for 24 h, 48 h and 72 h. At each time point images were collected by scanner (Epson, Japan). Colour change zones were measured from these images.

### EXAMPLES

### Preliminary Electrospinning

In order to find out the best electrospinning conditions for the polymer within Me₂CO₃ solutions, different PLC concentrations were tested (5% to 20%). It was seen that the best electrospinning was obtained with 15% PLC (w/w) in Me₂CO₃. Lower concentrations of PLC lowered the viscosity of the solutions and electrospraying tended to occur instead of electrospinning. For example, under the conditions of one experiment, 12.5% PLC fibres had beads on the fibre and fibres exhibited an inhomogeneous fibre diameter size distribution. The mean diameter of 12.5% PLC fibres using 15 kV was 5.38±4.78 μm. The chosen optimum concentration of 15% PLC resulted in electrospun fibres that had a mean diameter of 4.61±2.85 μm. Rather large fibre diameter was present, but the fibre diameter size distribution revealed that the fibres were with uniform size important for further testing.

The voltage effect was also studied. In one experiment, a range from 11 kV up to 15 kV was tested. The most suitable voltage for 15% PLC in Me₂CO₃ solution was found to be 13 kV with the apparatus used. It was seen that higher voltages tended to result in smaller and more homogeneous fibre diameters. However, when too great voltage was used, the spinning process became less continuous and electrospraying occurred.

### Testing Other Materials (Polymers, Solvents), and Conditions for In Situ Microfluidic Electrospinning

PLC in Me₂CO₃ formulation with agarose (concentrations ranging from 13% PLC to 15% PLC) as well as PLC/PEO in Me₂CO₃ formulation with agarose (concentrations ranging from 10% PLC+0.1% PEO to 13% PLC+0.3% PEO) were possible to be used for microfluidic electrospinning. In addition to the PLC/agarose and PLC/PEO/agarose microfluidic electrospinning, other formulations were also tested. Different polymers as well as solvents were tested which results are summarized here below.

PCL (Purasorb PC12 and PC08; polycaprolactone) with different molecular weights (80 000 vs 120 000) were tested (15% concentration) using Me₂CO₃ as a solvent, PCL was not dissolved within it, hence it was not possible to conduct microfluidic electrospinning. PDLA (Purasorb PDL 20; copolymer of DL-lactide) as another hydrophobic polymer was also tested in various concentrations. PDLA 15% in Me₂CO₃ dissolved but was too viscous for microfluidic electrospinning. PDLA 10% in Me₂CO₃ worked for electrospinning and could be tested for bacterial encapsulation. This polymer was not used for further testing but can be used according to the preliminary electrospinning experiments.

HFIP solvent was tested with PLC instead of Me₂CO₃, PLC 10% in HFIP microfluidics electrospinning was not possible which was confirmed by its unsuitability with the microfluidic chip (Table 1).

Different solvent mixtures were also tested together with Me₂CO₃, for example with propyl lactate, n-butyl lactate and diethylcarbonate. Solvent mixtures were used together with PLC 13.5% and Me₂CO₃, and the solvent ratios were (8.5:1)/Me₂CO₃: respective solvent. Diethyl carbonate was too viscous (needs lower PLC concentrations), but both propyl lactate and n-butyl lactate were nicely electrospinnable and no major “beads” or electrospinning errors were observed (Table 1).

TABLE 1
Compatibility of solvents with the polydimethylsiloxane (PDMS) microfluidic chip used for in situ microfluidic electrospinning.

| | | | | PDMS | | | | | | | |
|---|---|---|---|---|---|---|---|---|---|---|---|
| | | | | | mass | | | | | | |
| | | | | | after | | | | | | |
| | | | | | 24 h | | | | | | |
| solvent | | | | mass | with | | | | | | |
| | | volume | mass | before | solvent | swelling | swelling | water | PLC | | possibility |
| name | abbreviation | [ml] | [g] | [g] | [g] | [%] | factor | miscibility | solubility | remarks | of use |
| dimethyl carbonate | Me2CO3 | 3.000 | 3.200 | 0.152 | 0.175 | 15.1 | acceptable | − | + | | + |
| chloroform | CHCl3 | 3.000 | 4.120 | 0.149 | 0.451 | 202.7 | Severe | − | + | | + |
| hexafluoroisopropanol(1) | HFIP | 3.000 | 4.630 | 0.149 | 0.206 | 38.3 | noticeable | + | + | | − |
| dimethylformamide | DMF | 3.000 | 2.956 | 0.173 | 0.177 | 2.5 | negligible | + | − | | − |
| acetone | Me2CO3 | 3.000 | 2.180 | 0.148 | 0.185 | 24.7 | acceptable | + | − | | − |
| dimethylsulfoxide | DMSO | 3.000 | 3.356 | 0.201 | 0.206 | 2.4 | negligible | + | − | non | − |
| | | | | | | | | | | volatile | |
| dichloromethane | CH2Cl2 | 3.000 | 3.720 | 0.165 | 0.347 | 109.7 | strong | − | + | | + |
| ethyl acetate | CH3CO2CH3 | 3.000 | 2.660 | 0.198 | 0.321 | 61.5 | acceptable | − | − | | − |
| acetonitrile | CH3CN | 3.000 | 2.340 | 0.172 | 0.177 | 2.6 | negligible | ? | ? | toxic | ? |
| dichloroethane | CH2ClCH2Cl | 3.000 | 3.718 | 0.208 | 0.336 | 61.8 | strong | − | ? | toxic, | − |
| | | | | | | | | | | non | |
| | | | | | | | | | | volatile | |
| diethyl carbonate | Et2CO3 | 3.000 | 2.955 | 0.198 | 0.287 | 44.9 | acceptable | − | − | non | − |
| | | | | | | | | | | volatile | |
| n-butyl lactate | NBL | 3.000 | 2.966 | 0.148 | 0.165 | 11.5 | negligible | − | − | non | − |
| | | | | | | | | | | volatile | |
| propyl lactate | PL | 1.500 | 1.550 | 0.172 | 0.184 | 6.977 | negligible | ? | − | non | − |
| | | | | | | | | | | volatile | |

(1)time of contact with a solvent −48 hr

These data enabled to prove that also other hydrophobic polymers can be used (instead of PLC) for in situ microfluidic electrospinning. However, their suitability cannot be predicted without testing. Furthermore, the exact concentrations of polymers used in the formulation affect the results a lot. The selection of solvents is also very critical as suitable solvents need to be compatible with the microfluidic chip material, used polymers as well as living bacteria (without harming these during electrospinning).

### In Situ Microfluidics Electrospinning

Alternative formulations (sodium alginate together with CaCl₂) solution vs agarose aqueous solution) and concentrations of agarose aqueous solutions (0.625-2.5%) were tested in preliminary tests for microfluidics (data not shown). PLC 15% in Me₂CO₃ and 0.625% agarose in aqueous solution or PLC 12%+PEO 0.3% in Me₂CO₃ and 0.625% agarose were found to be the best combinations. PLC 15% in Me₂CO₃ and 0.625% agarose was selected as the initial test formulation for direct in situ microfluidics electrospinning to produce microcapsules and bacteria-loaded microcapsule fibre systems. The in-situ microfluidics electrospinning process was not affected when bacteria were incorporated into agarose aqueous solution. Different process parameters such as flow rate, voltage and distance were tested for in situ microfluidics electrospinning (Table 2).

Best processing conditions for in situ microfluidics electrospinning using the tested apparatus were: voltage of 15 kV, distance of 13 cm and flow rates varied from 0.4-0.5 ml/h for the polymer solution and 0.025-0.08 ml/h for the agarose and/or bacterial agarose dispersion. With other combinations of process parameters electrospinning was less effective. The main problem observed was the chips' channels getting blocked. It was observed that if the agarose channel speed was increased then the microcapsules were larger in size. If the size of the particles becomes too large this can make the electrospinning process unstable.

In situ microfluidics electrospinning of agarose microcapsules together with PLC in Me₂CO₃ solution resulted in fibres that were in some respects different compared to those generated from pristine (unloaded) PLC and Me₂CO₃ solution. It was observed that the obtained fibres were not so homogeneous, some larger diameter fibres were seen occasionally. However, the fibres had excellent mechanical properties.

In addition to these process parameters, also microfluidic PDMS chips with different designs were tested for microfluidics- and for the preparation of microcapsules (1×1×1 cm vs 2×1×1 cm vs 4×1×1 cm). From these tests it was seen that 1×1×1 cm microfluidic chip was the best using the conditions tested, as the formation of microcapsules was confirmed in microfluidics.

Microfluidics device allowed preparing agarose microcapsules prepared together with PLC and Me₂CO₃ solution with size ranging from 50-100 μm in diameter (when isolated in solvent), which can be directly electrospun if formulation composition, in situ microfluidics set-up and electrospinning conditions are optimized.

### In Situ Microfluidics Electrospinning to Prepare Living Bacteria-Loaded Fibres

Encapsulation of living bacteria via in situ microfluidics electrospinning was tested with different E. coli strains (E. coli MG1655, E. coli BW25113, E. coli Nissle) carrying different plasmids capable of producing different reporter proteins (mCherry, GFP, BFP). Main purpose of these experiments was to prove that bacteria can be encapsulated via in situ microfluidics electrospinning and these bacteria can be detected within electrospun fibres. Furthermore, that we do not see any differences with different E. coli strains (laboratory strain vs probiotic strain).

Microfluidics electrospinning was also performed with Lactococcus lactis strain IL1403. L. lactis is a Gram positive, lactic acid-producing bacterium widely used in food- and biotechnological processes and classified by the FDA as GRAS bacterium. Bacteria were pre-grown in M17 growth medium with 0.5% glucose. Initially, the same formulations and microfluidics (MF)/electrospinning (ES) conditions were used as for electrospinning of E. coli nissle, only different bacterium was used (L. lactis).

TABLE 2
Various formulations and processing parameters used
for in situ microfluidics electrospinning. Other environmental
and process parameters were kept constant (temperature,
humidity, needle size, syringe size).

| | Flow rate | Voltage | Distance |
|---|---|---|---|
| Formulations | (mL/h) | (kV) | (cm) |
| 15% PLC in Me₂CO₃ | Polymer: 1 | 15 | 15 |
| Agarose 0.625% | Agarose: 0.2 | | |
| 15% PLC in Me₂CO₃ | Polymer: 0.5 | 12 | 12 |
| Agarose 0.625% + | Agarose with | | |
| OD 1.10 | bacteria: 0.1 | | |
| E. coli BW25113 | | | |
| 15% PLC in Me₂CO₃ | Polymer: 1 | 12 | 13 |
| Agarose 0.625% + | Agarose with | | |
| OD 2.5 | bacteria: 0.1 | | |
| E. coli BW25113 | or 0.2 | | |
| 15% PLC in Me₂CO₃ | Polymer: 0.5 | 15 | 13 |
| Agarose 0.625% + | Agarose: 0.025 | | |
| E. coli BW25113 | | | |
| GFPmut2 | | | |
| 15% PLC in Me₂CO₃ | Polymer: 0.5 | 15 | 13 |
| Agarose 0.625% + | Agarose: 0.025 | | |
| E. coli MG1655 | | | |
| pC17_mCherry_lacl_tac_GFP | | | |
| 15% PLC in Me₂CO₃ | Polymer: | 15 | 13 |
| Agarose 0.625% + | 0.4-0.5 | | |
| E. coli Nissle | Agarose: | | |
| pSC101-Timer | 0.025-0.08 | | |
| tet0-BFP | | | |
| 15% PLC in Me₂CO₃ | Polymer: 0.6 | 15 | 13 |
| Agarose 0.625% + | Agarose: 0.05 | | |
| L. lactis | | | |

Control micrograph with labelled bacteria show the red fluorescence which allows the presence of bacteria to be detected (FIG. 3A). Micrographs reveal that one or more labelled bacteria can be placed into microcapsules during the encapsulation and some bacteria may also be within electrospun fibres separately from larger microcapsules, most likely having smaller coating around them for protection (FIG. 3).

FIG. 3 shows confocal fluorescent micrographs (CFM) of E. coli MG1655 pC17_mCherry_lacI_tac_GFP (A) and E. coli labelled with mCherry in PLC fibres after encapsulation by in situ microfluidics electrospinning. Images taken at different places on fibre mat (B, C, D).

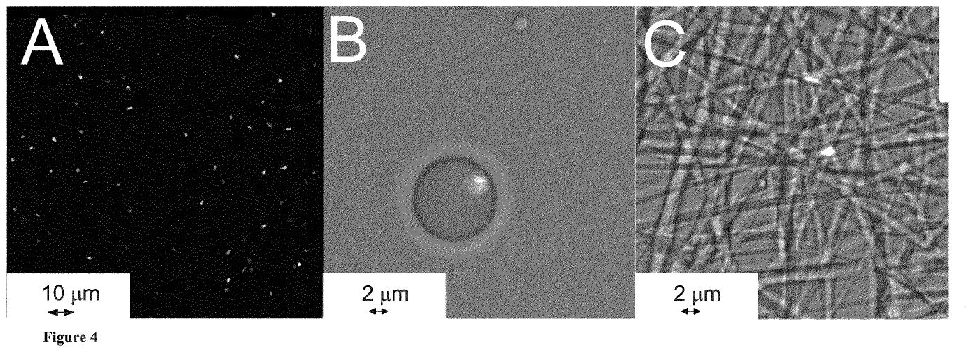

GFP-labelling enabled to find the bacteria within the microcapsules and also within electrospun fibres (FIG. 4). Both labels confirm that living bacteria were present and located within the microparticles and within the electrospun fibres.

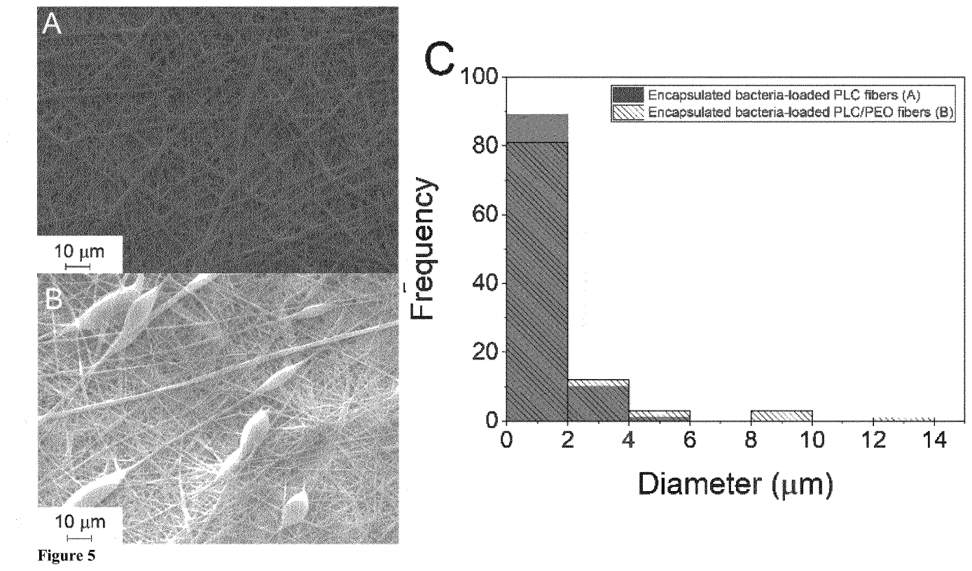

It was possible to obtain electrospun fibre matrices consisting of agarose microcapsule loaded Lactococcus bacteria in different formulation compositions (PLC vs PLC+PEO) (FIG. 5).

The mean fibre diameters were approximately 1.32±0.60 μm and 1.98±1.69 μm, for PLC and PLC/PEO formulations, respectively. Fibre diameter was more homogeneous for the PLC formulation compared to the PLC/PEO formulation, but the electrospinning process was better (constant flow without clogging) for the PLC/PEO formulation. The concentration of bacteria incorporated into microcapsule loaded electrospun fibres was the same for L. lactis (OD at 600 nm 2.0), as for the E. coli nissle strain. Viability and functionality of Lactococcus bacteria were tested with two different alternative methods: dead-live staining assay and developed pH reduction method.

### Morphology

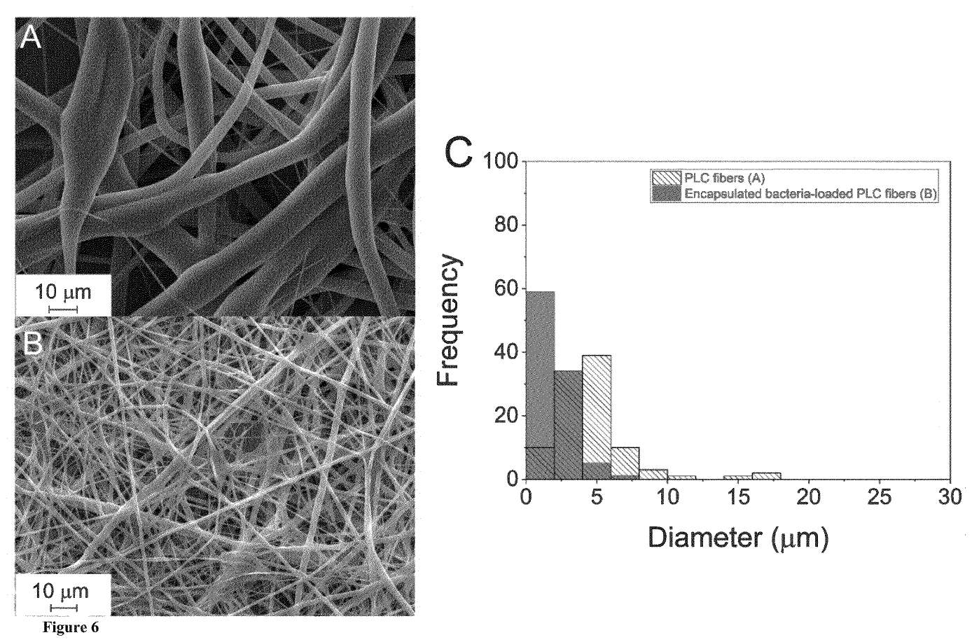

From SEM micrographs it was seen that the presence of agarose microcapsules together with encapsulated bacteria changed the morphology of electrospun PLC fibres (FIG. 6). Electrospun PLC fibres with E. coli BW25113 GFPmut2-loaded agarose microcapsules had a fibre diameter of 2.48±1.80 μm. Hence the fibre diameters were smaller compared to pristine PLC fibres (4.61±2.85 μm) prepared by monoaxial electrospinning. Fibre structure was also affected by the presence of bacteria-loaded microcapsules, in some places the fibre surface was porous and the presence of “beads” on the fibres was observed (FIG. 5). These beads could be the presence of microcapsules, which is also supported by CFM images (FIGS. 3 and 4). However, as also seen in CFM images, some microcapsules most likely cannot be detected in SEM micrographs, as the size of the microcapsules was similar to the fibre diameter. It is believed that most of the microcapsules are elongated within the fibres and cannot be distinguished in the SEM images.

FIG. 5 shows scanning electron microscopy (SEM) micrographs showing the morphology of pristine PLC fibres prepared by monoaxial electrospinning (A) and also PLC fibres including E. coli MG1655 pC17_mCherry_lacI_tac_GFP loaded agarose microcapsules prepared via in situ microfluidics electrospinning (B). A histogram showing the size distribution of the two fibre types is shown (C).

### Mechanical Properties

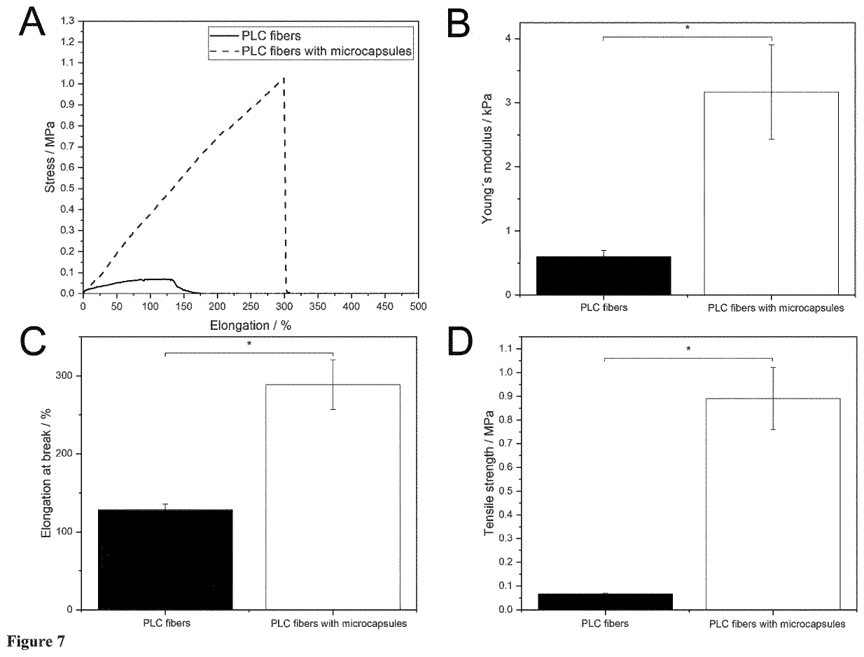

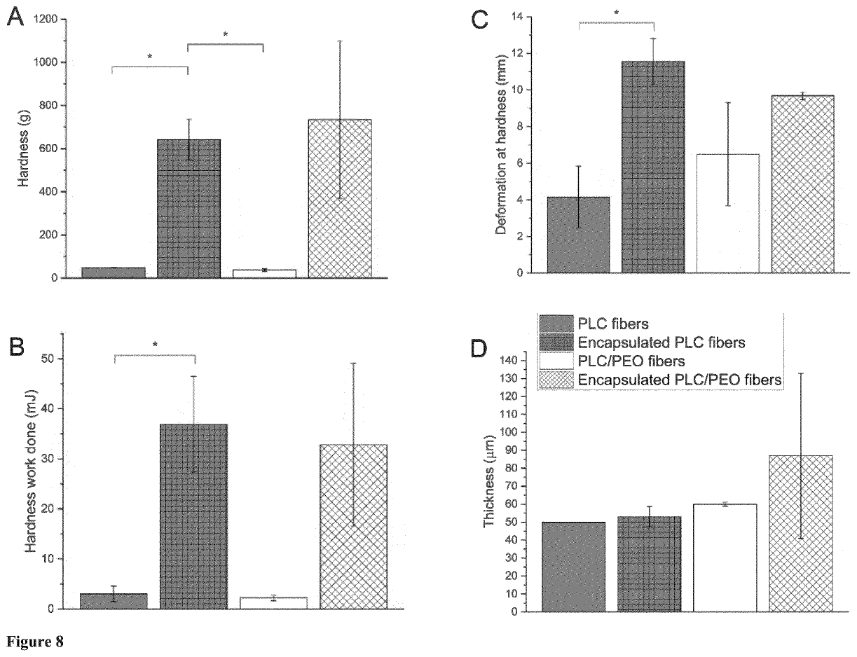

The mechanical properties of the fibre matrices are shown in FIG. 7 and FIG. 8. The data in FIG. 8 are a reanalysis of the raw data using different methodology to FIG. 7. It was surprisingly found that the incorporation of agarose microcapsules loaded with E. coli nissle pSC101-Timer tet0-BFP into the PLC fibres improved the mechanical properties of the matrix, having higher Young's modulus, tensile strength and elongation at break (higher elasticity of the fibre matrix) compared to pristine PLC fibres (FIG. 7). The Young's modulus was higher with PLC fibres loaded with EcN containing agarose microcapsules 3.17±0.74 kPa compared to pristine PLC fibres 0.60±0.10 kPa which indicates that the agarose microcapsule loaded PLC fibres with bacteria had higher stiffness (FIG. 7). Furthermore, the tensile strength was also higher with PLC fibres loaded with agarose microcapsules with EcN ranging from 0.76 to 1.02 MPa compared to pristine PLC microfibres with the tensile strength ranging from 0.06 to 0.07 MPa (FIG. 7). Prepared agarose microcapsule loaded PLC fibres with bacteria and agarose microcapsules loaded PLC/PEO fibres with bacteria had higher hardness values than their respective fibres without the microcapsules (FIG. 8). All the mechanical properties measured (hardness, deformation at hardness, and hardness work done) showed that the presence of microcapsule loaded bacteria within the fibres improved their mechanical properties (FIG. 8). This indicates that the produced PLC and PLC/PEO bacterial loaded agarose microcapsules within microfibres present a suitable platform for wound healing matrices due to the fact that these withstand higher forces caused by cell-fibre scaffold interactions.

### Swelling Behavior and Stability in Biorelevant Conditions

The behaviour of electrospun bacteria-consisting microcapsule loaded fibre matrices in biorelevant conditions (aqueous conditions and at 37° C.) gives important insight about their usability as wound dressings. Therefore, it is of importance to study the wettability (contact angle), swelling and degradation of these fibre mats.

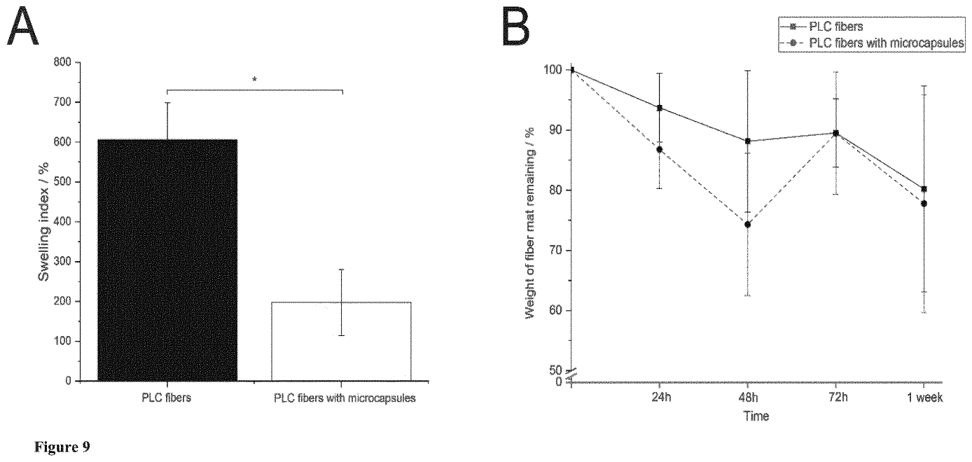

The swelling index for pristine PLC fibres was higher with the mean of 563.21±190.23% compared to the PLC fibres consisting of agarose microcapsules with encapsulated bacteria (EcN pSC101 Timer BFP) which mean value was 383.04±159.56% (FIG. 9A). The encapsulated bacteria-loaded PLC fibres showed higher weight loss (13.21±6.46%) compared to pristine PLC fibres (6.26±5.69%) after immersion into PBS for 24 h (FIG. 9B). Faster degradation of the mats is therefore expected for bacteria containing microcapsule loaded fibre mats compared to pristine PLC fibre mats. Degradation behaviour is of relevance when determining the exact time-period that the living bacteria-loaded electrospun fibre matrices can be used as dressings on the wound.

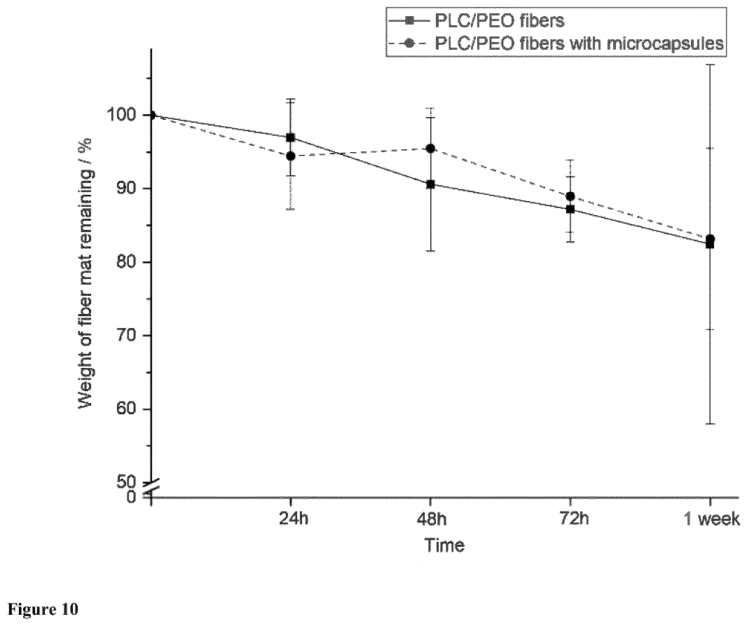

Prolonged degradation tests showed that both fibres (pristine and microcapsule loaded) had about 20% of weight loss during 1-week degradation testing (FIG. 9B and FIG. 10). According to the degradation test results, it is expected that the bacteria containing microcapsule are able to function in the wound as designed.

The contact angle measurements showed no statistical difference between the samples. Due to high contact angle values (around 120 to 140° for both mats loaded with microcapsules and “pristine” PLC unloaded fibre mats) the fibre mats have low wettability. The contact angle values were 89±18° and 121±13° for PLC/PEO fibres and microcapsule loaded PLC/PEO fibres, respectively. The latter affects the diffusion of active molecules out from the fibres and fibre mat and the diffusion of relevant substances into the fibres/fibre mats as well as fibre mat degradation behaviour. Furthermore, the contact angle values show that most likely eukaryotic cells are less prone to adhere on the mat surface.

SEM micrographs after the degradation test revealed that the morphology of the fibres was changed after being in contact with the aqueous medium for 1 week. When compared to the initial fibre diameters, then pristine PLC fibre diameters were reduced after being in contact with the aqueous medium (after the biodegradation test the fibre diameter was 3.62±2.30 μm compared to the initial fibre diameter of 4.61±2.85 μm). As expected from swelling index values after 24 h (FIG. 9A), it was evident that the swelling of fibres also occurred with PLC fibres in aqueous conditions in parallel with the degradation. Furthermore, encapsulated bacteria loaded PLC fibre diameters were significantly reduced (after the biodegradation test the fibre diameter was 1.02±0.33 μm compared to initial fibre diameter of 2.48±1.80 μm). Fibre size distribution analyses revealed that unloaded PLC fibres had a more heterogeneous fibre size from 1 μm up to 4 μm, whereas microcapsule loaded PLC fibres had smaller fibre diameter, approx. 1 μm and more homogeneous fibre size distribution. In addition to the changed fibre diameters, also the fibre surface was covered with small particles and broken fibres which were not detected with initial dry fibre matrices.

It was also visually verified that the mats changed their appearance, for example pristine PLC fibres were reduced in size-contracted (2×2 cm mat was 1×1 cm mat after being in contact with aqueous conditions).

### Safety of Electrospun Bacteria-Loaded Electrospun Fibres (Biomat)

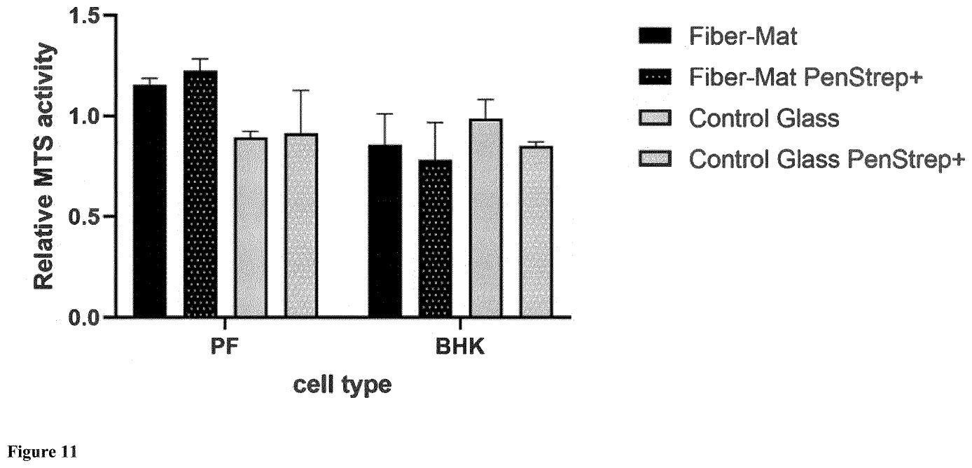

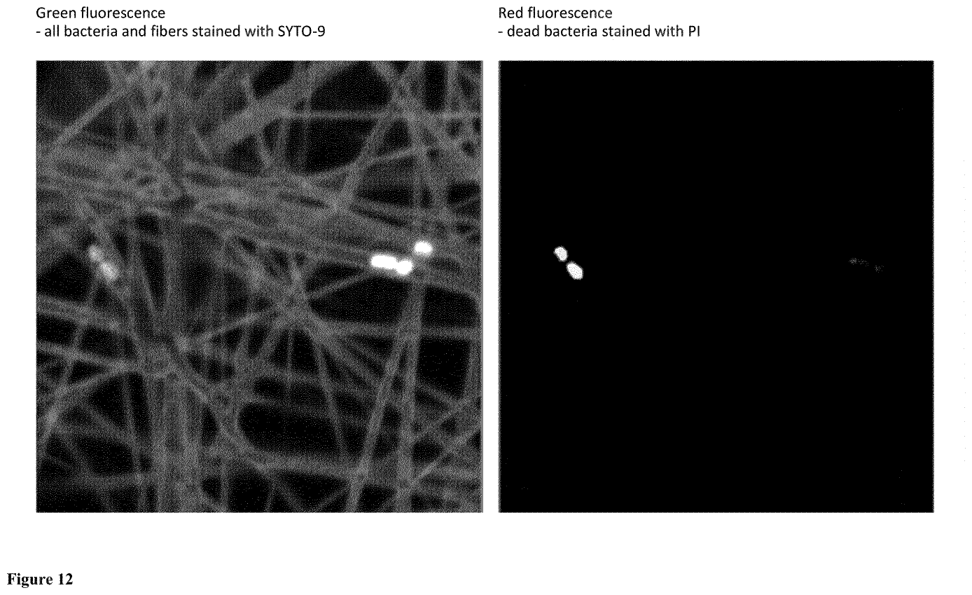

Mammalian cell viability evaluation was performed with fibroblast cells grown on electrospun L. lactis bacteria-loaded fibre mats. The results showed that cell viability was not reduced compared to the controls (cells on glass slides) (FIG. 11). In addition, growth media with and without antibiotic supplement (PenStrep+) was compared. Results on FIG. 11 show that there is no difference in fibroblast viability whether the bacteria in the fibres are alive, or their growth and survival have been inhibited by the antibiotics (PenStrep+). The results demonstrate that both fibre matrix materials and loaded bacteria are biocompatible with relevant skin cells and this enables these fibre mats to be used as wound dressings.

### Functionality of Electrospun Bacteria-Loaded Fibres (Biomat)

Continuous electrospinning was possible with the formulation which consisted of 12% PLC+0.3% PEO in Me₂CO₃, and microfibers were obtained with this formulation and bacteria were located within these fibres. When this 12% PLC+0.3% PEO/0.625% agarose+OD2.0 bacteria formulation and optimized microfluidic and electrospinning conditions were used, the prepared electrospun fibres consisted living L. lactis bacteria and their viability and functionality were proven also using dead-live assay and developed pH reduction evaluation method.

Dead-live staining assay was used to confirm the viability of bacteria within the electrospun fibres as well as to show the diffusion of substances (SYTO9) into the fibres and staining of dead bacteria with PI (FIG. 12).

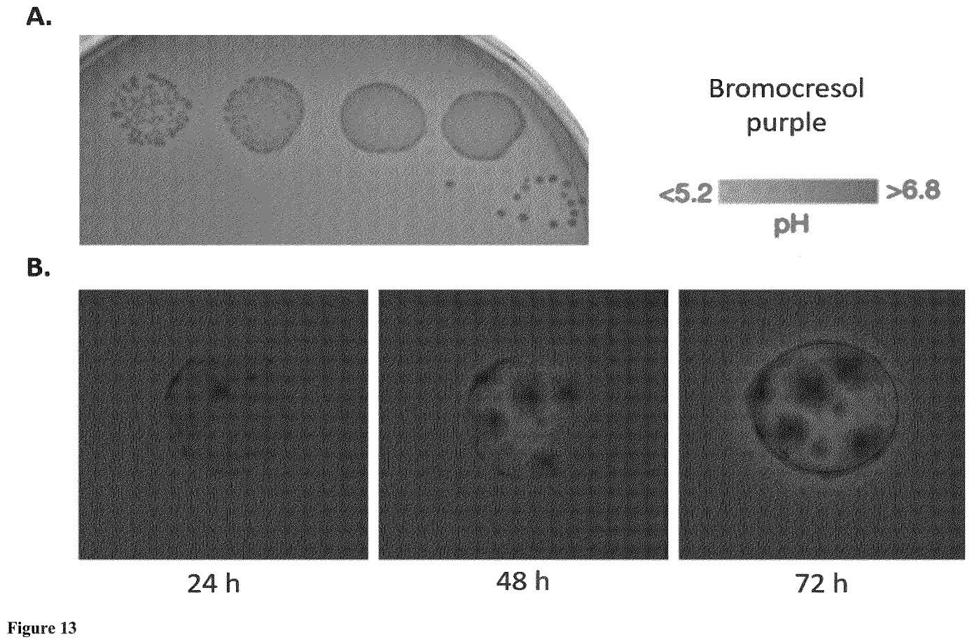

Alternatively, to prove the viability and functionality, the production of bacterial metabolite (e.g. lactic acid) was measured when Lactococcus was grown on glucose (as described in the literature DOI: 10.1126/scitranslmed.aao2586). L. lactis bacteria, including laboratory 1403 strain, produces lactic acid which lowers the pH of the surrounding environment. The lowering of pH enables to determine the viability and metabolic activity of L. lactis within the electrospun fibres and the change of colour enables to prove that lactic acid is released from fibres. This pH reduction agar plate diffusion method allows to visualize the viability and functionality of L. lactis bacteria within microcapsules and electrospun fibres for longer time-periods (prolonged activity). The pH change close to viable L. lactis bacteria (producing lactic acid) was determined using pH indicator Bromocresol purple (Fisher scientific, Thermo Fischer, USA) (4.6 mg of indicator in 200 mL medium) which changes its colour in different pH conditions (from purple to yellow in acidic environment) (FIG. 13A). The higher the bacterial concentration, the more visible is the yellow colour surrounding the bacteria.

The method revealed that L. lactis bacteria produce lactic acid within microcapsules and microcapsule-loaded electrospun fibres already after 24 h, this metabolite lactic acid is released into the surrounding environment as yellow circles were visible surrounding the electrospun fibre matrices (FIG. 13B). The colour change close to the electrospun fibre mat confirms that pH change is detected. After 48 h and 72 h the size of changed colour zone increased together with increased intensity of yellow colour confirming the viability of bacteria and their functionality to produce lactic acid.

### Viability of Bacteria

One of the most important part of the study was to confirm the viability of bacteria within electrospun PLC fibres and determine for how long the bacteria are viable and metabolically active. Furthermore, it is important to get an overview whether molecules are able to diffuse through the matrix. Two-way transport is needed: relevant substances for bacteria are able to diffuse into the fibres and relevant substances produced by bacteria are able to diffuse out from the fibres. Usually fibres are dissolved in a buffer and then analysed by CFU counting if hydrophilic polymer matrices are used. However, in case of hydrophobic polymer matrices different approaches need to be used. For this purpose E. coli nissle bacterium was prepared incorporating the pSC101-Timer-TetR-PtetA-BFP-noDeg plasmid. The viability and metabolic activity of the cells can be determined either from the intensity of the blue signal from BFP or the green/red signal ratio from TIMER. Dividing cells have higher green inensity signal and non-dividing or dormant cells have both signals-green and red. The green/red signal ratio helps to determine the cellular behavior.

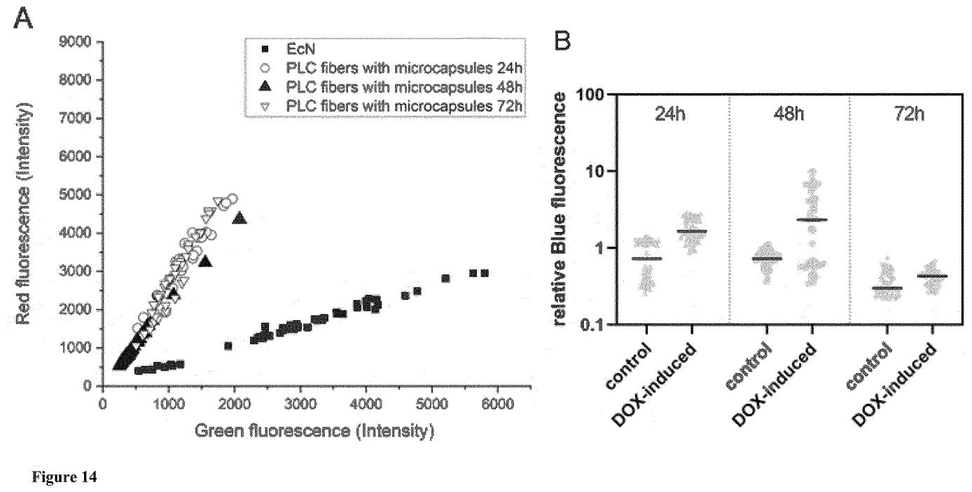

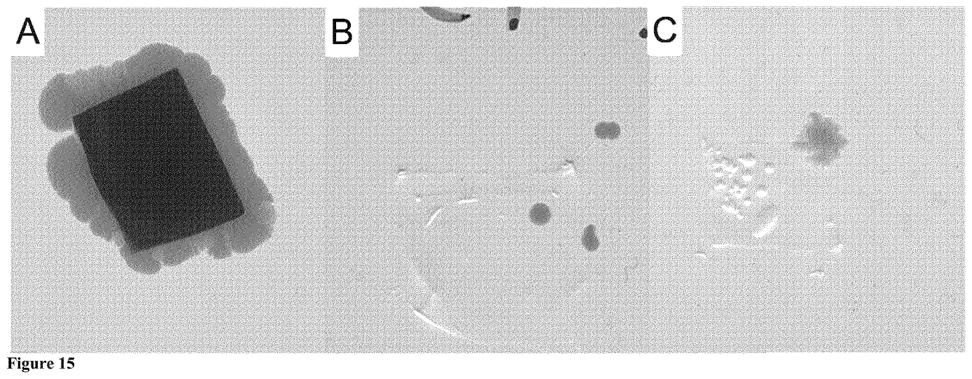

It was seen that bacteria prior electrospinning were very viable and had a high green/red signal ratio. With electrospun samples we saw increase in the intensity of this signal from Oh to 48 h timepoints, followed by decrease after 72 h. The highest viability was observed at 48 h timepoint, where about 50% of the bacteria were alive. This correlates to the BFP signal seen on FIG. 14. The bacteria are capable of producing BFP only in the presence of inducer (doxycyclinehen the inducer was added, it was possible to detect the bacteria together with BFP labelling within the fibres using CFM. These results confirm that bacteria loaded into microcapsules and into fibres are viable and metabolically active. Futhermore, it was confirmed that the inducer is able to diffuse into the fibres and therefore also the diffusion in and out from the fibres is possible. The tested example mats are able to work efficiently up to 72 h, after which time it may be desirable to renew the fibre mat for wound and/or infection treatment applications utilising live bacteria.

### Encapsulation Efficiency and Purity

In addition to the viability of bacteria, the encapsulation efficiency and purity of the bacteria-loaded electrospun fibre matrices are important parameters that need to be tested in order to understand the possible level of leaching of bacteria from the fibres. Encapsulation of bacteria into microcapsules and into the fibres prolongs the action time of the bacteria in the wound dressing and potentially aids control of drug release where an API is encapsulated. It is desirable to keep the number of viable bacteria within the wound dressing constant or within an active window for the usage time of a wound dressing.

The experiment revealed a clear bacterial growth confirming that even though most of the bacteria were nicely encapsulated into the PLC fibres during in situ microfluidics electrospinning, there were some bacteria not incorporated and able to leach. These tests confirmed a small leaching of bacteria from fibres.

FIG. 15A shows the leaching of E. coli nissle pSC101-Timer tet0-BFP from PLC fibres with microcapsules after 24 h of incubation on LB agar plates (fibre mat on folio upside on the plate). Growth is seen only from the corners from a small number of cells present outside of the fibres, FIGS. 15B and 15C show the leaching of E. coli nissle pSC101-Timer tet0-BFP from PLC fibres with microcapsules after 48 h of incubation on LB agar plates where the fibre mat with folio was removed after 24 h of incubation. It was seen that bacteria also leach out from other places of the mat, but can be detected when sample is removed.

Since in the test case these are non-GMO and probiotic bacteria, it is not a problem for wound healing application. Rather, these probiotic bacteria are known to have immunomodulatory activity (Salva, Villena and Alvarez, 2010) and have shown to enhance the wound healing (Lukic et al., 2017; Sinha et al., 2019).

In the event that non-encapsulated bacteria or other bioactive entities were undesirable, an additoinal washing step can be used. An additional washing step was added prior the use of bacteria-loaded fibre mats on the wound, in order to confirm whether the number of non-encapsulated bacteria can be reduced.

The Examples demonstrate that a hydrophobic polymer solution in organic solvent can be used as a continuous phase for the preparation of bioactive entity-containing microparticles in a microfluidic device and these can be directly electrospun into fibres. No intermediate steps were required between the microfluidic encapsulation step and the electrospinning step.

Microfluidics is a promising method for encapsulating bioactive entities, including living bacteria. The present inventors have shown that microfluidics can be efficiently combined with electrospinning technique in order to have in situ electrospinning of bioactive entities, including living bacterial cells.

Release of Substances from Electrospun Fibres and Diffusion into the Fibres

The release of substances from microcapsules and electrospun fibres consisting of microcapsules enables to prove the transport of substances in and out of the microcapsules and microcapsule-loaded fibres. It is the prerequisite for providing conditions for bacteria to survive and also to collect the substances produced by bacteria within such systems. Two different methods were used for the analysis: 1) release of blue food dye (Blue sky, UK) (preliminary testing) and 2) diffusion of stains (SYTO9 and PI) into and out of the fibres and staining of bacteria.

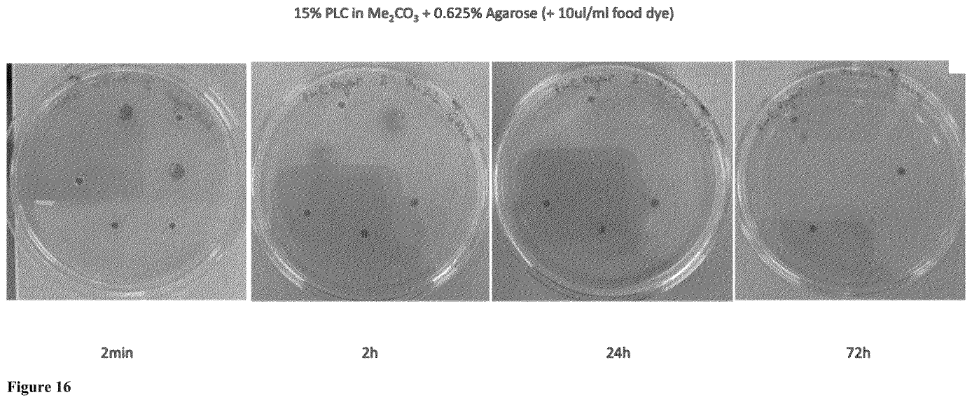

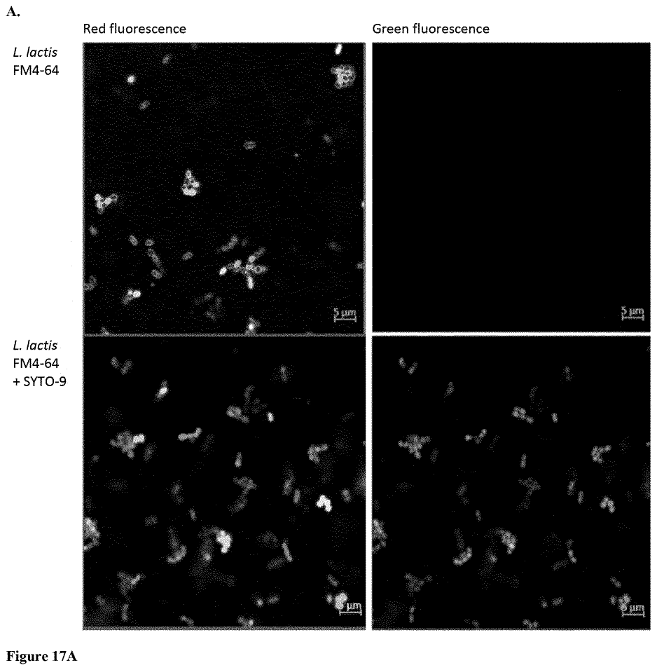

Initially, the release of blue food dye from the microcapsules and microcapsule-loaded fibres was tested. Microcapsules consisting of 15% PLC in Me₂CO₃+0.625% agarose (+10 μL/mL food dye) were prepared, collected on LB agar plate and the release of dye was investigated (FIG. 16). The droplet was released from the syringe tip when approximately 10 microcapsules were formed and formed a larger droplet. 6 separate samples were collected on LB plates. Approximately after 2 min, the diffusion of blue food dye was observed with 2 samples, the other 4 samples remained unchanged due to the formation of PLC film between agar and microcapsule droplet not allowing food dye to diffuse (FIG. 16).

LB plates were stored at 37° C. and after 2 h two more samples had released the dye; the others samples remained the same. After 24 h of incubation, the food dye had diffused altogether from 5 samples. After 72 h of incubation all samples had released some amount of blue food dye.

This method confirms that formed microcapsules in a microfluidic device (prior electrospinning) can release the substances from their interior. The release is dependent on the contact between microcapsule and formation of PLC film, the latter somewhat inhibits the release of substances. The release of blue food dye into the buffer solution was also tested with electrospun microcapsule-loaded fibres (15% PLC in Me₂CO₃+0.625% agarose (+10 μL/mL food dye). For this experiment, the electrospun fibre samples were put into the buffer solution and the release of blue food dye from electrospun fibre mats was measured by measuring the absorbance at 410 and 630 nm. It was seen that blue dye was released very slowly from the mat into the buffer solution within the tested timeframe.

PEO as a hydrophilic polymer was used to increase the nanoporosity and consequently the diffusion of molecules through the fibres. Formulation composition was modified by adding PEO as a porogen into the PLC fibres. Different formulations were tested by varying the PLC as well as PEO concentrations and ratios. Addition of PEO directly to PLC 15% solution resulted in too viscous solutions, and electrospinning was not possible (tested 0.5%, and 0.1% PEO addition). Concentrations varying between PLC 10%+PEO 0.1% in Me₂CO₃ and PLC 13%+0.3% PEO in Me₂CO₃ worked for microfluidic electrospinning. It was visually observed that the addition of PEO within the PLC layer enabled to increase the release of blue dye from the microcapsule loaded electrospun fibres.

### MF/ES Conditions:

Flow rate for PLC: 0.1 mL/h
Flow rate for agarose: 1.0 mL/h
Needle: 21G (microfluidic needle)
Distance between the needle and collector: 13 cm
Voltage: 15.0 kV
RH: 22% (used dehumidifier)
Temperature: 33° C.
Volume of electrospun bacterial dispersion: 30, 50 or 100 μL (depending on the samples collected).

### Formulation:

12% PLC+0.3% PEO in Me₂CO₃+0.625% agarose (with bacteria OD2.0) was further used for testing the diffusion of substances, viability and functionality of the bacteria-loaded fibre matrices.

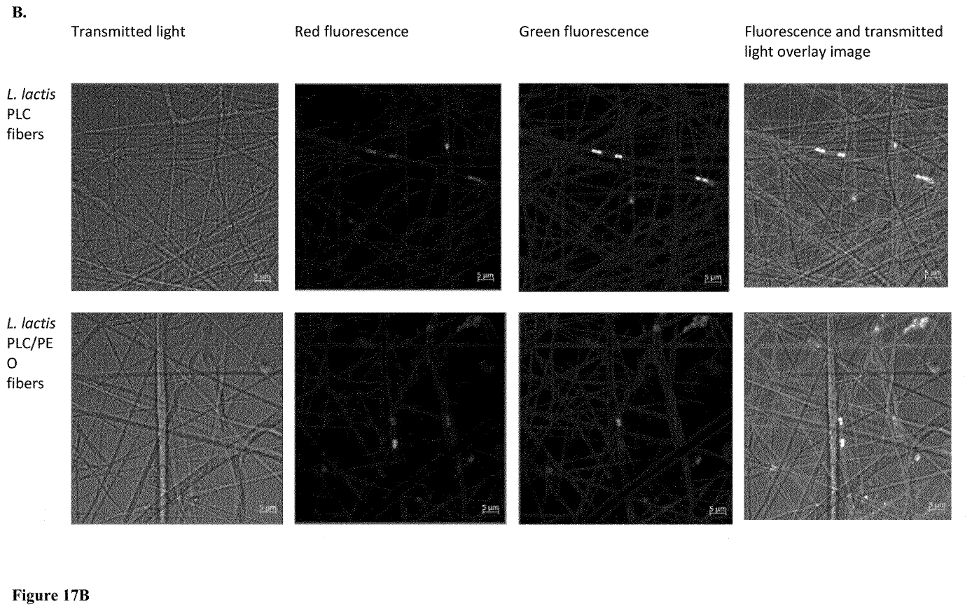

The staining of bacteria within the fibres with SYTO-9 demonstrated that the stain can diffuse into fibres. We evaluated the diffusion of green-fluorescent nucleic acid stain SYTO-9 into the microcapsule loaded PLC/PEO fibres and its capability to stain nucleic acid of bacteria (appearance of green fluorescence) (FIG. 17). Bacteria were initially (before electrospinning) stained using red-fluorescent membrane stain FM-4-64. The visualization was performed using confocal fluorescent microscopy. Control bacteria (free bacteria outside the fibres) were imaged before and after staining with SYTO-9 (FIG. 17A). The results show that all bacteria stained with FM-4-64 resulting with red fluorescence but no detectable green fluorescence (FIG. 17A), but when SYTO 9 was added, the bacteria became also green-fluorescent (FIG. 17B). SYTO-9 staining of the electrospun PLC or PLC/PEO fibres containing FM4-64 pre-stained L. lactis showed that SYTO 9 stain was able to diffuse through the fibres and microcapsules and stain nucleic acid of most of the bacteria (FIG. 17B). However, in PLC fibres and in lesser extent in PLC/PEO some fibre-encapsulated bacteria were not stained with SYTO-9.

Different assays were developed and used to prove the functionality of bacteria-loaded electrospun fibres prepared by in situ droplet microfluidics electrospinning summarised in the following Table 3.

TABLE 3
Summary of the designed and used assays to investigate the functionality
of electrospun bacteria-loaded fibre matrices.

| Description of assay | | | Assay designed for testing | |
|---|---|---|---|---|
| | Bacteria/ | | Perme- | Viability |
| | active | Additional | ability | and func- |
| | substances | staining | of fibres | tionality |
| Detection | inside | of fibre | to small | of bacteria |
| method | fibres | mats | molecules | in fibres |
| Microscopy | E. coli | — | NA | YES |
| | expressing | | | |
| | fluorescent | | | |
| | proteins | | | |
| | L. lactis | SYTO-9 | YES | NA |
| | pre-stained | | | |
| | with FM4-64 | | | |
| | L. lactis | SYTO-9/PI | YES | YES |
| Plate | L. lactis | — | YES | YES |
| diffusion | metabolizing | | | |
| | glucose | | | |
| | to produce | | | |
| | lactic acid | | | |
| | Blue food dye | — | YES | NA |
| | CAM | — | YES | NA |

Key: CAM—chloramphenicol; NA—non-applicable.
Assays coloured in grey enable to study both the permeability and diffusion of substances, as well as viability and functionality of bacteria.

All the assays give complementary results enabling to prove the functionality of electrospun bacteria-loaded fibres. Indeed, not all assays enable to study both aspects: permeability/diffusion of substances and viability and functionality of bacteria within the fibres (Table 3). Only microscopy with dead-live staining with SYTO 9/PI and PH reduction agar diffusion assay provide evidence for both relevant aspects.

### Drug Substance Encapsulation (Electrospinning of Drugs Instead of Bacteria)

It was also tested if unstable substances can be electrospun using the microfluidic electrospinning method (for example antibiotics, antimicrobial peptides, AMPs). Tests were performed with antibiotic agents chloramphenicol (CAM) and antimicrobial peptide D-pleurocidin (D-Pleu). The electrospinning and encapsulation of CAM and D-Pleu into the agarose microcapsule-loaded fibres were successful for both substances.

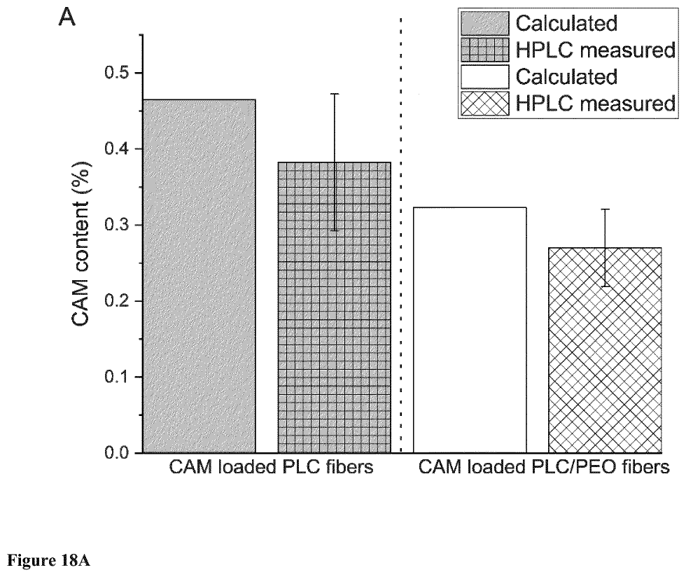

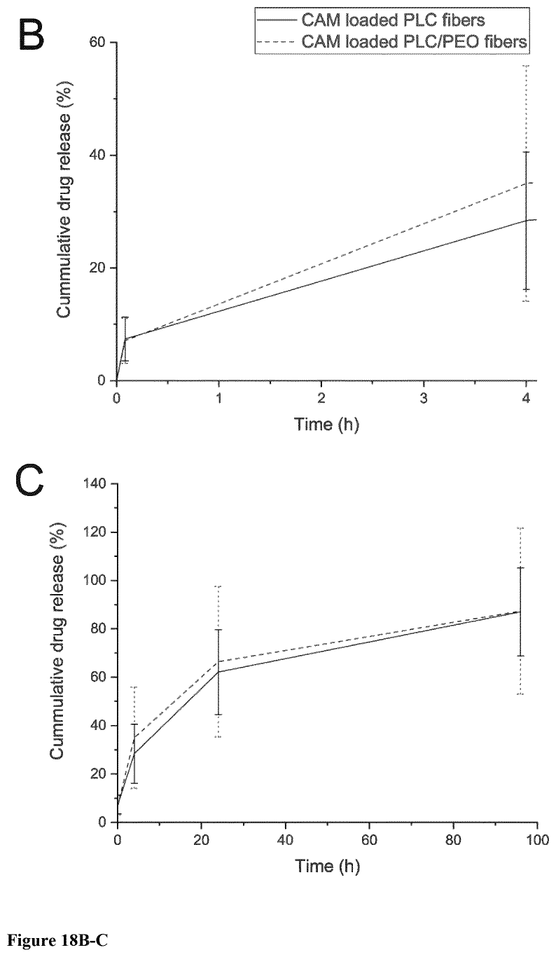

Initially, PLC 15% in Me₂CO₃ and agarose 0.625% were used and CAM was included in the agarose phase. Secondly, PLC 12%/0.3% PEO in Me₂CO₃ and 0.625% agarose formulation was electrospun. The concentration of CAM within the microcapsule loaded electrospun PLC and PLC/PEO fibres was confirmed by HPLC matching with the theoretical CAM contents (FIG. 18A). Exact electrospinning conditions used for the experiments are provided in the Materials and Methods section and using these electrospinning conditions resulted in approximately 0.120-0.160 g of the produced mat. Electrospinning of 12% PLC+0.3% PEO formulation required slight modifications in parameters (s.f. Materials and Methods section), but similarly to the bacteria-loaded fibres, this PLC/PEO formulation was better processable for microfluidics electrospinning than pristine PLC one.

It was seen that a prolonged release of CAM was obtained when CAM was included into the microcapsules and fibres during microfluidic electrospinning (FIGS. 18B and C). The incorporation of PEO into the formulation did not modify the initial burst release (compared to the pristine PLC formulation), which was expected as PEO needs to be dissolved before nanopores can be formed in aqueous environment (FIG. 18B). However, the presence of PEO and its fast solubility did not affect also the CAM release from PLC/PEO fibres in comparison with the PLC fibres in later time-points (FIG. 18C). The latter is most likely due to the major differences between the samples (N=3) as a result of folded fibre samples in the vials during the drug release assays. Larger matrices were prepared during electrospinning and the diffusion of CAM out from the folded fibre samples affected the results much more compared to the nanoporosity. Hence the nanoporosity and its effect on the drug release was superimposed by the fibre sample macroscale differences. Therefore, the expected higher release rates of CAM from PLC/PEO fibre mats remain expected but were not proven.

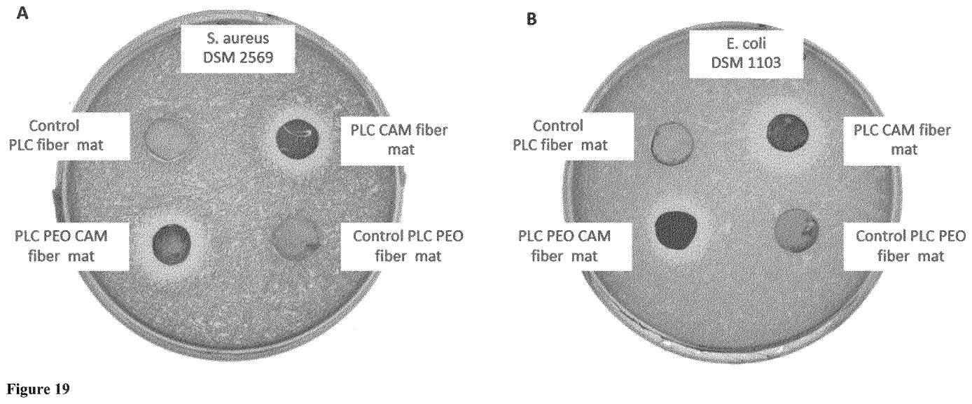

The CAM-loaded fibre matrices also provided good antibacterial activity as proven using agar diffusion assay with relevant pathogenic wound bacteria (E. coli and S. aureus isolates) (FIG. 19).

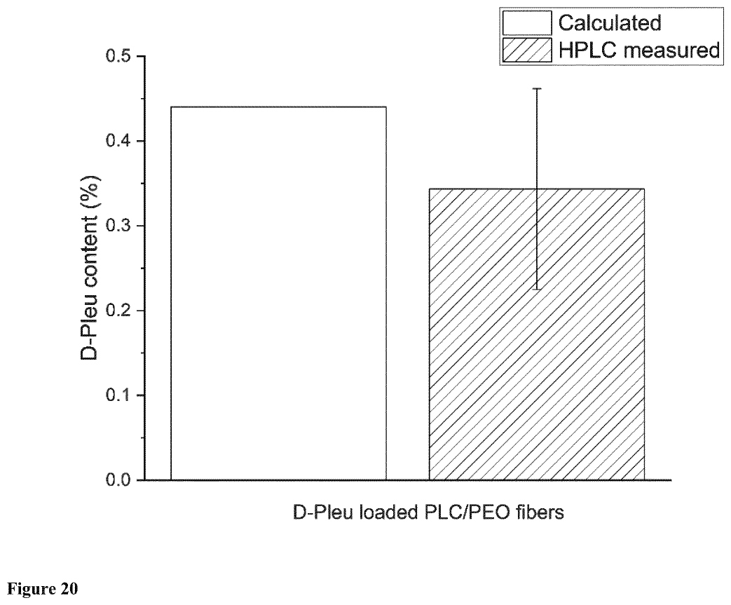

Incorporation of unstable antimicrobial peptide D-Pleu which is known to be difficult to be electrospun by using the conventional monoaxial electrospinning method due to its fast degradation, was also successful with in situ droplet microfluidic electrospinning. The measured drug content within electrospun fibres was approximately 0.34%+0.12% matching with the theoretical concentration value of 0.44 (FIG. 20). Therefore, almost 78% of D-Pleu was present within the fibres after in situ microfluidic electrospinning without major degradation which takes place during conventional monoaxial electrospinning of D-Pleu and resulting in 0% of measured D-Pleu content. The latter shows that the method is suitable to be used for the electrospinning of unstable drug molecules such as antimicrobial peptides and the preparation of novel drug delivery systems (DDSs).

1. A method for producing electrospun polymer fibres comprising bioactive entity-containing microparticles, the method comprising the steps:
(a) encapsulation of a bioactive entity via microfluidics; and
(b) electrospinning into polymer fibres,
wherein encapsulation and electrospinning are performed using a single apparatus.

2. The method of claim 1, wherein the bioactive entity is a cell or active pharmaceutical ingredient (API).

3. The method of claim 1 or 2, wherein the bioactive entity comprises bacteria, yeast, fungi or mammalian cells.

4. The method of any of claims 1 to 3, wherein the output of encapsulation step a) is the input of electrospinning step b).

5. The method of any of claims 1 to 4, wherein the encapsulated bioactive entities are electrospun without any filtration, drying or purification steps between steps a) and b).

6. The method of any preceding claim, wherein the continuous phase is a hydrophobic polymer solution in organic solvent.

7. The method of claim 6, wherein the hydrophobic polymer is a copolymer of L-lactide and ε-Caprolactone (PLC).

8. The method of claim 6 or claim 7, wherein the organic solvent comprises or consists of dimethyl carbonate.

9. The method of claim 8, wherein the concentration of PLC in dimethyl carbonate is 11 to 20% (w/w), preferably 15% (w/w).

10. The method of claim 6, wherein the continuous phase comprises or consists of PLC and poly(ethylene oxide) (PEO) in dimethyl carbonate.

11. The method of claim 10, wherein the concentration of PLC in dimethyl carbonate is 10 to 20% (w/w), preferably 12% (w/w) and the concentration of PEO in dimethyl carbonate is 0.1 to 1.0% (w/w), preferably 0.3% (w/w).

12. The method of any of claims 1 to 11, wherein the bioactive entities are encapsulated in hydrophilic and/or gel-forming polymer microparticles.

13. The method of claim 12, wherein the hydrophilic and/or gel-forming polymer is agarose.

14. The method of any of claims 1 to 13, further comprising:
(c) washing the polymer fibres to remove bioactive entity not located within the fibres.

15. The method of any preceding claim, wherein the bioactive entity comprises bacteria, and wherein the bacteria produce a desired substance.

16. The method of claim 15 wherein the desired substance is an antimicrobial substance, such as antimicrobial peptides and/or antibiotics.

17. The method of any of claims 1 to 16, wherein the electrospun polymer fibres are used for wound healing and/or infection treatment application.

18. The method of any of claims 1 to 16, wherein the electrospun polymer fibres are used for the treatment of acne, atopic dermatitis or scars.

19. Electrospun polymer fibres comprising bioactive entity-containing hydrophilic and/or gel-forming polymer microparticles embedded within fibres of at least one hydrophobic polymer.

20. The electrospun polymer fibres of claim 19 formed or formable according to the method of any of claims 1 to 18.

21. The electrospun polymer fibres of claim 19 or claim 20 containing at least 1.0E+05 viable bacteria per mm³.

22. The electrospun polymer fibres according to any of claims 19 to 21, wherein the bacteria are capable of producing at least one desired substance, such as an antimicrobial substance.

23. The electrospun polymer fibres according to any of claims 19 to 22 for use in wound healing and/or infection treatment.

24. The electrospun polymer fibres according to any of claims 19 to 22 for use in the treatment, prevention or reduction of acne, atopic dermatitis or scars.

25. A wound dressing comprising electrospun polymer fibres of any of claims 19 to 22.

26. An apparatus comprising a microfluidic device and an electrospinning device comprising at least two devices inducing flow, a microfluidic device, a nozzle, a high-voltage power supply, a grounded collection plate and an electrospinning device, wherein the microfluidic device comprises at least two input channels and at least one output channel, wherein the first liquid is an aqueous solution containing bioactive entity, the second input liquid is a polymer solution in organic solvent, the microfluidic device is configured such that the two input liquids introduced to dedicated input channels meet and generate a polymer solution comprising entrained microcapsules of bioactive entity, which pass to the nozzle, and the nozzle is held at a potential of +3-60 kV or −3-60 kV relative to the grounded collection plate.
